(19) United States
(12) Patent Application Publication
(10) Pub. No.: US 2014/0170108 A1
(43) Pub. Date: Jun. 19, 2014

(54) COMPOSITIONS USEFUL FOR THE TREATMENT OF INFLAMMATORY DISEASE OR DISORDERS

(71) Applicant: NATIONAL INSTITUTE OF IMMUNOLOGY, New Delhi (IN)

(72) Inventors: Avadhesha Surolia, New Delhi (IN); Shweta Pasi, New Delhi (IN); Sarika Gupta, New Delhi (IN)

(21) Appl. No.: 14/118,603

(22) PCT Filed: May 18, 2012
(86) PCT No.: PCT/IB2012/000975

§ 371 Date: Feb. 11, 2014

Related U.S. Application Data
(60) Provisional application No. 61/577,793, filed on Dec. 20, 2011.

(30) Foreign Application Priority Data
May 19, 2011 (IN) 3014/DEL/2010

(51) Int. Cl. A61K 38/17; C07K 16/24; A61K 45/06; A61K 38/16

(52) U.S. Cl. CPC A61K 38/1793; A61K 38/16; C07K 16/241; A61K 45/06
USPC 424/85.2; 530/402; 514/21.2; 514/16.6; 424/133.1; 424/142.1

(57) ABSTRACT

The present invention provides sustained release and long acting forms of peptide therapeutic, particularly Interleukin-1 receptor antagonist (IL-1ra), including multimeric forms of IL-1ra, including variants of IL-1ra which are capable of multimerising, and compositions comprising the long acting and multimeric forms of IL-1ra, and a process of preparation thereof. The present invention also provides compositions comprising the multimeric forms of IL-1ra, including IL-1raK, KIL-1ra and KIL-1raK, which are effective in inhibiting, treating and/or ameliorating rheumatoid disease, inflammatory diseases or disorders, autoinflammatory disorders or conditions resulting from adverse effects of Interleukin-1 (IL-1). Methods of treating a subject comprising administering the composition comprising the multimeric forms of IL-1ra are also provided.

### CROSS REFERENCE TO RELATED APPLICATIONS

The present application is a National Stage Application claiming the priority of PCT Application No. PCT/IB 12/00975 filed May 18, 2012, which in turn, claims priority from U.S. Provisional Application Ser. No. 61/577,793 filed Dec. 20, 2011 and from Indian Application 3014/DEL/2010 filed May 19, 2011. Applicants claim the benefits of 35 U.S.C. §120 as to the PCT Application and priority under 35 U.S.C. §119 as to the said U.S. Provisional application and Indian Application, and the entire disclosures of both applications are incorporated herein by reference in their entireties.

### FIELD OF THE INVENTION

The present invention relates to sustained release and long acting forms of peptide therapeutic, particularly Interleukin-1 receptor antagonist (IL-1ra), including multimeric forms of IL-1ra, including variants of IL-1ra which are capable of multimerising, and compositions comprising the long acting and multimeric forms of IL-1ra. The multimeric IL-1ra and long acting, sustained release compositions are effective in inhibiting, treating and/or ameliorating inflammatory diseases or disorders, rheumatoid disease, autoinflammatory disorders, immune disorders, autoimmune disorders, and/or diseases or conditions resulting from adverse effects or activities of Interleukin-1 (IL-1).

### BACKGROUND OF THE INVENTION

Proteins, with their dynamic and diverse physiological roles as macromolecules, constitute a class of therapeutics that came into existence over 20 years ago with the use of the first recombinantly produced protein, therapeutic Insulin. The protein therapeutics subset of therapy has grown immensely in number and use, with hundreds of molecules approved or in development. Some of the qualities that make these biologics treatments of choice over traditional chemical agents or small molecules include their non-interference with normal biological processes, low immunogenicity, and better tolerance in an animal. However, these remarkable properties are over-shadowed by limitations such as low in vivo stability and short plasma half life, which contribute to poor bio-availability and hence low efficacy of these molecules. Constrained by these issues, efficacy enhancing compensatory measures often result in high, frequent and multiple dosing along with high peaks or low levels of the biopharmaceutical, translating into unwanted side-effects or limited therapeutic benefit. Therefore, enhancing the in vivo efficacy and sustainability of biological therapeutics is still a challenge.

Inflammation is a biological response of the host to a harmful stimulus which may be external or internal such as pathogens, necrosed cells and tissues, irritants etc. Inflammation, though, protective in nature can sometimes become abnormal and result in self tissue injury and may lead to various diseases and disorders such as asthma, glomerulonephritis, inflammatory bowel disease, rheumatoid arthritis, hypersensitivities, pelvic inflammatory disease, autoimmune diseases, etc. Therefore, active termination of harmful inflammatory responses is of utmost importance for protection against unnecessary tissue and organ damage.

Rheumatoid arthritis (RA) is a chronic, systemic, inflammatory disorder of autoimmune origin. The disease is characterized by inflammation of joints particularly, synovial membrane, cartilage and bone, leading to irreversible joint damage with eventual loss of function and deformity. It is estimated to affect 0.5-1% of world population with significant morbidity and mortality. Though arthritis causes fewer deaths as compared to cancer and cardiovascular diseases, there is no other group of diseases that causes so much of suffering in so many people for prolonged durations.

Studies elucidating the pathogenic mechanisms associated with synovitis and articular damage have helped to gain insights into the autoimmune processes of the disease which are driven by autoreactive T and B-lymphocytes, both of which produce pro-inflammatory mediators. Increased expression and functional activity of cytokines, particularly of Interleukin-1 (IL-1) and TNF-α, has been found in the rheumatoid synovial fluid and tissues (Feldmann M et al (1996) Annu Rev Immunol 14:397-440).

Evidences from animal studies have established the role of IL-1 as the major contributor to the disease process in RA. IL-1β induces arthritis when injected directly into murine joints (Pettipher E R et al (1986) Proc Natl Acad Sci USA 83:8749-53). Both IL-1α and IL-1β have been shown to induce bone and cartilage destruction in murine antigen-induced arthritis (van de Loo F A J et al (1995) Am J Pathol 146:239-49).

Increased systemic levels of IL-1β have been detected in patients with RA (Chikanza, I. C. et al (1995) Arthritis Rheum 38:642-648) and these levels were found to have a correlation with disease severity (Eastgate, J. A. et al (1988) Lancet 2:706-708; Rooney, M. et al (1990) Rheumatol Int 10:217-219). Elevated levels of IL-1β have been detected in the synovium, synovial fluid and cartilage of RA patients (Firestein, G. S. et al (1992) Arthritis Rheum 149:1054-1062).

Inflammatory bowel disease (IBD) is a multifactoral inflammatory disorder of the gastrointestinal tract. It has two clinically distinct forms namely Crohn's disease and ulcerative colitis (UC) which affect either the entire gastrointestinal tract or specifically the colonic mucosa manifesting as chronic remittent or chronic progressive conditions. It is a serious health problem affecting 1 in 1000 individuals in the western world. The symptoms of the disease include abdominal pain, persistent diarrhea, anorexia, weight loss and intestinal ulceration which can result into death under extreme circumstances. Disease pathogenesis in IBD is an outcome of a complex interplay between several factors such as genetic factors, intestinal flora, environmental factors such as misuse of antibiotics, diet, hygiene, stress, etc and the host immune system. Dysregulated immune response resulting in a cellular milieu rich in activated immune cells and proinflammatory cytokines, a hallmark of any inflammatory condition, is also a common feature of IBD. Increased expression of certain pro-inflammatory cytokines such as IL-1, TNF-α, IL-6, IL-8, etc has been found in the intestinal mucosa of patients suffering from IBD. These proinflammatory cytokines recruit the blood-borne effector cells by activating the endothelium to upregulate adhesion molecules and release chemokines.

IL-1, the classic mediator of inflammation, plays a significant role in mucosal inflammation. The interleukin-1 receptor antagonist (IL-1ra) is a member of the IL-1 family and binds to the IL-1 receptor, but does not induce an intracellular response. IL-1ra was initially called the IL-1 inhibitor and was identified as a native protein in mammals (Liao Z et al (1984) J Exp Med 159(1):126-136; Liao Z et al (1985) J Immunol 134(6):3882-3886). U.S. Pat. No. 6,599,873 describes human IL-1ra. IL-1ra prevents IL-1 from sending a signal and inhibits the activities of IL-1 alpha and IL-1 beta. Endogenous IL-1ra is produced in numerous experimental animal models of disease and in human autoimmune and chronic inflammatory diseases (Arend W P et al (1998) Ann rev Immunol 16:27-55). Mucosal biopsies from IBD patients show increased expression of IL-1β in comparison to IL-1 receptor antagonist (IL-1ra) (Casini-Raggi V et al ( ) J Immunol 154:2434-40). Neutralization of IL-1β by anti-IL-1β antibody or administration of IL-1ra has been shown to ameliorate colitis in an animal model (Cominelli F et al ( ) J Clin Invest 86:972-80), while neutralization of IL-1ra had destructive effects.

Of the several approaches being used to target the IL-1 pathway, IL-1 Receptor Antagonist (IL-1ra) is the only FDA approved drug currently in clinical practice (Kineret® (anakinra)). Anakinra is a recombinant non-glycosylated form of human IL-1 ra that differs from native IL-1ra by addition of a single methionine residue at its N-terminus. Anakinra is approved for clinical use in rheumatoid arthritis (RA), used as a monotherapy or in combination with one or more disease-modifying anti-rheumatic drugs (DMARDs). The drug was tested for use in RA patients, particularly those non-responsive or poorly responsive to other DMARDs, such as TNF antibody (infliximab (Remicade) or adalimumab (Humira) (Fleischman R A et al (2003) Arthritis & Rheumatism 48(4):927-934; Nuki G et al (2002) Arthritis & Rheumatism 46(11):2838-2846; Cohen S et al (2002) Arthritis & Rheumatism 46(3):614-624). The usual dosage is 100 mg subcutaneously once a day. Anakinra is administered as a daily injectable, however, in spite of a daily dosing regimen IL-1ra is limited in its efficacy in the treatment of RA because of its short biological half life of only 4-6 hours. Indirect data suggests that anakinra may be inferior to TNF-α inhibitors as currently formulated. The plasma half life of anakinra ranges from 4-6 hours after subcutaneous administration at clinically relevant dose of 1-2 mg/kg (kineret-eu.com).

Therefore, there remains a need to provide an effective treatment for inflammatory diseases or disorders, rheumatoid disease, auto inflammatory disorders or conditions resulting from adverse effects of Interleukin-1 (IL-1) and to provide alternative, effective and longer-lasting forms of IL-1ra.

The citation of references herein shall not be construed as an admission that such is prior art to the present invention.

### SUMMARY OF THE INVENTION

In accordance with the present invention, novel forms of peptide therapeutic are provided which are multimeric and which release active monomers of the peptide therapeutic in a sustained manner. The multimeric forms of peptide therapeutic of the present invention demonstrate longer biological half life and release active peptide monomers over sustained periods in vivo. Such multimeric forms are generated or achieved by attaching a multimerising motif to the therapeutic peptide, thereby conferring the capability for effective and useful multimerisation to the therapeutic peptide. The multimerising motif confers biologically relevant and useful character and capability to a therapeutic peptide, particularly wherein monomer therapeutic peptide, or peptide without a multimerising motif, has a short half life in vivo, requires daily or regular administration because of half life or stability, is unstable in vivo, and/or forms inactive aggregates in vitro or in vivo. The multimeric forms of therapeutic peptide of the invention provides an alternative long acting, stable and active form of the peptide therapeutic, with enhanced and/or useful capability in vivo. The enhanced and/or useful capability of the multimeric forms of therapeutic peptide of the invention includes one or more of increased half-life, increased stability in vivo, sustained release of active monomeric peptide over days. In an aspect of the invention, the multimerising motif may confer active multimerization to a peptide therapeutic, particularly wherein monomeric peptide aggregates or forms inactive or less active aggregates or forms in the absence of the multimerising motif. The multimeric compositions of the invention, including as provided herein, release monomers of peptide therapeutic, in a sustained manner for a long or extended period resulting in improved therapeutic efficacy in terms of reduction or amelioration of associated disease parameters and circumventing the need of administration of the peptide therapeutic drug on daily basis.

In a particular aspect of the invention, novel forms of IL-1 antagonist, particularly of IL-1ra, are provided which are multimeric and which release active monomers of IL-1ra in a sustained manner. The multimeric IL-1ra forms provide long-acting, useful and effective IL-1ra capable of inhibiting, treating and/or ameliorating IL-1 mediated or associated disease, including rheumatoid disease, acute and chronic inflammatory diseases or disorders, autoinflammatory disorders or conditions resulting from adverse effects of Interleukin-1 (IL-1), including rheumatoid arthritis (RA), Inflammatory Bowel Disease (IBD), Ulcerative colitis (UC), and acute hepatic injury.

The multimeric peptide forms of the present invention comprise modified or variant peptide therapeutics having one or more multimerising motif attached, wherein the multimerising motif confers active aggregation, enabling formation of aggregates of the peptide therapeutic. The aggregates release active monomeric therapeutic peptide over sustained periods, particularly over days or weeks, thereby providing sustained release forms or formulations and compositions of a peptide therapeutic. The multimerising motif may be covalently attached to a peptide monomer. The multimerising motif may be covalently attached to peptide at the N-terminus, at the C-terminus, or at the N- and C-terminus of a therapeutic peptide. A concatamer peptide is also contemplated comprising one or more multimerising motif and one or more peptide monomer in a single peptide. The multimerisation motif of use in the present invention may be selected from one or more of KFFE, KVVE, KFFK, EFFE, GNNQQNY, KLVFFAE, NGAIL, NFLV, FLVHS, NFGSVQFV, DFNKF and DFNK or an active multimeric variant thereof.

In an aspect of the invention, a multimeric variant of IL-1ra is provided comprising IL-1ra having one or more multimerising motif covalently attached to monomeric IL-1ra, wherein the multimeric variant forms aggregates of Il-1ra peptide capable of releasing active IL-1ra monomers over a sustained period in vivo. The multimeric IL-1ra of the present invention comprises modified or variant IL-1ra, which provides a form or composition of IL-1ra that releases active monomers of IL-1ra in a sustained manner over an extended period. Multimeric IL-1ra comprises IL-1ra that has a multimerising motif attached thereto, thereby conferring multimerising capability to the IL-1ra. The multimerising motif may be covalently attached to IL-1ra peptide at the N-terminus, at the C-terminus, or at the N- and C-terminus thereof. The multimeric compositions of the invention, including IL-1raK, KIL-1ra and/or KIL-1raK compositions as provided herein, release monomers of variant IL-1ra, such as IL-1raK or KIL-1ra or KIL-1raK, in a sustained manner for a long or extended period resulting in improved therapeutic efficacy in terms of reduction such IL-1 associated disease parameters as pain and inflammation, thus circumventing the need of administering the IL-1ra drug on daily basis.

The present invention provides a composition for enhancing the in vivo shelf life and in turn/thereby efficacy of protein, peptide or small molecule therapeutics. The composition described in the present invention comprises of incorporating a multimerising motif, such as KFFE, into a protein, peptide or small molecule that leads to molecular clustering and formation of a depot at the site of injection. The present invention uses IL-1 receptor antagonist (IL-1ra) to demonstrate the utility of these multimerising motifs.

Thus, while normal human or recombinant IL-1ra exists as a monomer and as a biological response modifier agent is administered as a daily injectable (for example Kineret® (anakinra)), the multimeric IL-1ra of the present invention provides an IL-1ra composition which releases active monomers and thereby provides a sustained release farm of IL-1ra, releasing active and monomeric IL-1ra. The multimeric IL-1ra of the invention is useful in any applications or indications for which IL-1ra is already applicable and in clinical practice or evaluation. In addition, due to the long acting and sustained release parameters of the multimeric IL-1ra of the invention versus native monomeric IL-1ra, the multimeric IL-1ra of the invention has applications and uses for which monomeric native IL-1ra is less effective, including because of the short half life in vivo of monomeric native IL-1ra.

In one embodiment the present invention discloses multimeric forms of variants of IL-1 receptor antagonist (IL-1ra), wherein the variant is IL-1 receptor antagonist comprising a multimerisation motif covalently attached at the C terminus, N terminus, or at the C and N termini which are effective in inhibiting, treating and/or ameliorating IL-1 mediated diseases or conditions, including rheumatoid disease, acute and chronic inflammatory diseases or disorders, autoinflammatory disorders or conditions resulting from adverse effects of Interleukin-1 (IL-1), rheumatoid arthritis (RA), Inflammatory Bowel Disease (IBD), Ulcerative colitis (UC), and acute hepatic injury. In particular embodiments, multimeric forms of variants of IL-1ra are provided wherein the variant is IL-1 receptor antagonist comprising the multimerisation motif KFFE (SEQ ID NO:18) at C terminus (IL-1raK), IL-1 receptor antagonist comprising KFFE at N terminus (KIL-1ra) or IL-1 receptor antagonist comprising KFFE at C and N termini (KIL-1raK), which are effective in inhibiting, treating and/or ameliorating rheumatoid disease, acute and chronic inflammatory diseases or disorders, autoinflammatory disorders or conditions resulting from adverse effects of Interleukin-1 (IL-1), rheumatoid arthritis (RA), Inflammatory Bowel Disease (IBD), Ulcerative colitis (UC), and acute hepatic injury. In alternative embodiments, multimeric forms of variants of IL-1ra are provided wherein the variant is IL-1 receptor antagonist comprising the multimerisation motif selected from one or more of KVVE (SEQ ID NO:19), KFFK (SEQ ED NO:20) and EFFE (SEQ ID NO:21), covalently attached at the C terminus, N terminus, or at the C and N termini for use in inhibiting, treating and/or ameliorating IL-1 mediated diseases or conditions. In further embodiments, multimeric forms of variants of IL-1ra are provided wherein the variant is IL-1 receptor antagonist comprising the multimerisation motif selected from one or more of GNNQQNY (SEQ ID NO:22), KLVFFAE (SEQ ID NO:23), NGAIL (SEQ ID NO:24), NFLV (SEQ ID NO:25), FLVHS (SEQ ID NO:26), NFGSVQFV (SEQ ID NO:27), DFNKF (SEQ ID NO:28) and DFNK (SEQ ID NO:29), covalently attached at the C terminus, N terminus, or at the C and N termini for use as provided.

The present invention provides exemplary multimeric forms of variants of IL-1 receptor antagonist (IL-1ra), wherein the variant is IL-1 receptor antagonist comprising KFFE at the C-terminus, N-terminus, and the C- and N-terminus, having amino acid sequence as set forth in any of SEQ ID NOs: 1-3 respectively. Alternative variants having one or more amino acid substitutions in the IL-1ra native sequence and comprising one or more multimerisation motif are further contemplated, wherein they possess the multimerisation and sustained release and log acting characteristics or the variants disclosed and described herein.

The multimeric form of variants of IL-1ra, such as IL-1raK, KIL-1ra, KIL-1raK, have morphology similar as depicted in FIG. 2C, whereby the multimeric form appears to be a result of non-covalent interactions between individual protein molecules by way of the multimerising motif (KFFE for instance), whereby the aggregates appear as protein sticks bundled together, wherein each stick consists of linear arrays of individual protein molecules. The tertiary structure of individual protein molecules appears to be conserved during the multimerisation process of the inventors as it is aided by the multimerisation motif, for example KFFE.

In an additional aspect of the present invention there is provided a process of preparation of multimers of IL-1 receptor antagonist (IL-1ra) (SEQ ID NO: 4) or active sequence variants or allelic variants thereof. In an aspect thereof is provided a process of preparation of multimeric form of IL-1ra variant, including IL-1raK, KIL-1ra, KIL-1raK, the process comprises dissolving the IL-1ra variant, such as IL-1raK, KIL-1ra, KIL-1raK, at a temperature of 25-50° C. in a solution having a pH in the range of 4-8 and incubating the above for a period of 6-48 hours with constant shaking to obtain multimeric form of the variant. The multimeric IL-1ra variants generated via the process of the invention are effective in inhibiting, treating and/or ameliorating rheumatoid disease, acute and chronic inflammatory diseases or disorders, autoinflammatory disorders or conditions resulting from adverse effects of Interleukin-1 (IL-1), rheumatoid arthritis (RA), Inflammatory Bowel Disease (IBD), Ulcerative colitis (UC), and acute hepatic injury. Another embodiment of the present invention provides a process of preparation of multimeric IL-1ra, wherein the process comprise dissolving IL-1ra having attached multimerisation motif at a temperature of about 25-50° C. in a solution having a pH in the range of about 4-8 and incubating the above for a period of about 6-48 hours with constant shaking to obtain multimeric IL-1ra, wherein multimeric IL-1ra comprises insoluble multimers of IL-1ra variants.

In another embodiment of the present invention there is provided a composition comprising multimeric forms of IL-1raK, KIL-1ra, KIL-1raK, wherein the composition is useful for in inhibiting, treating and/or ameliorating rheumatoid disease, acute and chronic inflammatory diseases or disorders, autoinflammatory disorders or conditions resulting from adverse effects of Interleukin-1 (IL-1), rheumatoid arthritis (RA), Inflammatory Bowel Disease (IBD), Ulcerative colitis (UC), and acute hepatic injury.

The present invention further includes a composition comprising multimeric forms of IL-1ra variant formed by expressing these proteins as fusion proteins with a multimerising motif at C, N or both termini, which are effective in inhibiting, treating and/or ameliorating rheumatoid disease such as arthritis. In an aspect, the invention includes a composition comprising multimeric forms of IL-1ra variant formed by expressing these proteins as fusion proteins with a multimerising motif at C, N or both termini, which are effective in inhibiting, treating and/or ameliorating IL-1 mediated disease(s) or condition(s), wherein the multimerisation motif is selected from KFFE, KVVE, KFFK and EFFE. The present invention provides in an exemplary aspect a composition comprising multimeric forms of IL-1raK, KIL-1ra, KIL-1raK formed by expressing these proteins as fusion proteins with the multimerising motif KFFE at C, N or both termini, which are effective in inhibiting, treating and/or ameliorating rheumatoid disease such as arthritis. In a further aspect, the invention includes a composition comprising multimeric forms of IL-1ra variant formed by expressing these proteins as fusion proteins with a multimerising motif at C, N or both termini, which are effective in inhibiting, treating and/or ameliorating IL-1 mediated disease(s) or condition(s), wherein the multimerisation motif is selected from one or more of GNNQQNY, KLVFFAE, NGAIL, NFLV, FLVHS, NFGSVQFV, DFNKF and DFNK, covalently attached at the C terminus, N terminus, or at the C and N termini for use as provided. The composition may comprise a multimeric IL-1ra variant comprising an amino acid sequence selected from SEQ ID NOs: 1-3 and 5-16, or may comprise a multimeric IL-1ra variant comprising the IL-1ra sequence as set out in SEQ ID NO:4 covalently attached to a multimerisation motif selected from one or more of KFFE, KVVE, KFFK, EFFE, GNNQQNY, KLVFFAE, NGAIL, NFLV, FLVHS, NFGSVQFV, DFNKF and DFNK or an active multimeric variant thereof.

Still another embodiment of the present invention provides a composition comprising insoluble and multimeric forms of IL-1ra, including IL-1raK, KIL-1ra, KIL-1raK or a combination thereof, wherein the composition is useful as a protein therapeutic for the treatment of autoinflammatory disorders selected from the group consisting of arthritis, rheumatoid arthritis (RA), Inflammatory Bowel Disease (IBD), Ulcerative colitis (UC), and acute hepatic injury. Still another embodiment of the present invention provides a composition comprising insoluble and multimeric forms of IL-1raK, KIL-1ra, KIL-1raK or a combination thereof and any other drug or compound useful for the treatment of inflammatory diseases or disorders, wherein the composition is useful as a protein therapeutic for the treatment of autoinflammatory disorders selected from the group consisting of arthritis, rheumatoid arthritis (RA), Inflammatory Bowel Disease (IBD), Ulcerative colitis (UC), and acute hepatic injury.

The invention provides methods of amelioration, treatment and/or inhibition of IL-1 mediated disease, including rheumatoid disease, arthritic conditions, inflammatory conditions, and immune conditions, whereby multimeric IL-1ra is administered. In one such aspect of this method a variant IL-1ra is administered which is capable of sustained release of IL-1ra monomers, such that the variant IL-1ra provides a long acting form of IL-1ra. In a particular aspect, the multimeric IL-1ra administered releases active monomers of IL-1ra over a period of at least 1 day, 2 days, 3 days, 5 days, 7 days, more than 3 days, more than 5 days, more than 7 days. The rheumatoid disease may be selected from a group consisting of arthritis, Ankylosing Spondylitis, Avascular Necrosis, Osteonecrosis, Behcet's Syndrome, Bursitis, Cervical Spondylosis, Fibromyalgia, Dupuytren's Disease, Gout, Infectious Arthritis, Neurogenic Arthropathy, Osteoarthritis, Pseudogout, Psoriatic Arthritis, Polymyalgia Rheumatica, Giant Cell Arthritis, Reiter's Syndrome (Reactive Arthritis), Rheumatic Fever and Rheumatic Heart Disease, Rheumatoid Arthritis, Scleroderma, Sjögren's Syndrome, Still's Disease, Systemic Lupus Erythematosus, Tendinitis Arthritis/Tendonitis Arthritis, Vasculitis, Muckle-Wells syndrome, Wegener's Granulomatosis and multiple sclerosis. Other conditions may be selected from multiple sclerosis, graft-versus-host disease, prevention of acute graft rejection, sarcoidosis, systemic lupus erythematosus, giant-cell arteritis, inflammatory bowel disease, ulcerative colitis, Crohn's disease, malignancies that require IL-1 as a mitogen such as solid tumors or leukemia, HIV-related Kaposi's sarcoma, uveitis, neonatal onset multisystem inflammatory disease, psoriasis, adverse cardiac remodeling after acute myocardial infarction, sepsis, tumor-mediated immune suppression.

The invention provides a method for treating and/or ameliorating rheumatoid disease, acute and chronic inflammatory diseases or disorders, autoinflammatory disorders or conditions resulting from adverse effects of Interleukin-1 (IL-1), rheumatoid arthritis (RA), Inflammatory Bowel Disease (IBD), Ulcerative colitis (UC), and acute hepatic injury, wherein the method comprises administering to a subject in need thereof a therapeutically effective amount of the composition comprising variants of IL-1ra or combination thereof at a dose which is effective for the alleviation of the disorder, wherein the variant is a multimeric IL-1 ra, such as IL-1raK, KIL-1 ra, or KIL-1raK.

In another of the embodiment there is provided a method of treating, inhibiting, and/or ameliorating inflammatory diseases or disorders, rheumatoid disease, autoinflammatory disorders or conditions resulting from adverse effects of Interleukin-1, said method comprises administering a therapeutic amount of the multimeric form of IL-1ra, such as IL-1raK, KIL-1ra and KIL-1raK, as disclosed in the present invention to a subject in need and further administering an additional therapeutic agent, in combination, simultaneously, concurrently, or separately.

In a further aspect, the present invention provides use of multimeric forms of variants of IL-1ra or combination thereof for treating and/or ameliorating rheumatoid disease, acute and chronic inflammatory diseases or disorders, autoinflammatory disorders or conditions resulting from adverse effects of Interleukin-1 (IL-1), rheumatoid arthritis (RA), Inflammatory Bowel Disease (IBD), Ulcerative colitis (UC), and acute hepatic injury, wherein the variant is IL-1raK, KIL-1ra, KIL-1raK, IL-1raKVVE, KVVEIL-1ra, KVVEIL-1raKVVE, IL-1raKFFK, KFFKIL-1ra, KFFKIL-1raKFFK, IL-1 raEFFE, EFFEIL-1ra, or EFFEIL-1raEFFE, or combinations thereof.

Still yet another embodiment of the present invention provides the composition of multimeric IL-1 raK in combination with multimeric KIL-1ra for the treatment of inflammatory and autoinflammatory disorders selected from the group consisting of arthritis, Inflammatory Bowel Disease (IBD), Ulcerative colitis (UC), acute hepatic injury. The present invention provides the use of multimeric IL-1raK in combination with multimeric KIL-1ra for the treatment of autoinflammatory disorders selected from the group consisting of rheumatoid arthritis.

The present invention also provides a multimerising motif that is incorporated at C-terminus, N-terminus, at both termini or is generated by modification of residues within the protein or peptide sequence. The present invention provides a multimerising motif containing hydrophobic residues at any position of which at least one is aromatic. The present invention also provides a multimerising motif containing hydrophobic residues that acquire β-conformation. The present invention further provides a multimerising motif containing residues with complementary charges at any position which are capable of co-polymerising. The present invention also provides a multimerising motif harboring an α-helical conformation capable of generating β-strands.

The present invention provides a composition comprising multimeric forms of variants (such as exemplary IL-1raK, KIL-1ra, KIL-1raK) of IL-1 receptor antagonist (IL-1ra) formed by expressing these proteins as fusion proteins with one or more multimerising motif at C, N, or both termini. The present invention also describes the inhibition, treatment, and/or amelioration of acute and chronic inflammatory, autoinflammatory, metabolic, neurodegenerative, malignant and other acute and chronic diseases/disorders by these fusion proteins in mammals, in particular, human subjects.

In another embodiment of the present invention there is provided a composition comprising multimeric IL-1ra, as exemplified by IL-1raK, useful as protein therapeutics for the treatment of inflammatory and autoinflammatory disorders selected from the group consisting of arthritis, Inflammatory Bowel Disease (IBD), Ulcerative colitis (UC), acute hepatic injury, in human subjects, wherein the said composition comprises of insoluble multimers of IL-1raK. In yet another embodiment of the present invention there is provided a composition in the form of multimeric IL-1 raK useful as protein therapeutics for the treatment of autoinflammatory disorders selected from the group consisting of rheumatoid arthritis, in human subjects, wherein the said formulation comprises insoluble multimers of IL-1raK.

In an aspect hereof, the present invention provides a composition comprising multimeric form peptide therapeutic, wherein the multimeric form releases active monomer peptide therapeutic for at least 2 days, at least 3 days, at least 4 days, at least 5 days, at least 6 days, at least 7 days, at least a week, up to 2 days, up to 3 days, up to 4 days, up to 5 days, up to 6 days, up to 7 days, up to 8 days, up to 6±2 days in vitro or in vivo.

One embodiment of the present invention provides a composition comprising multimeric IL-1ra, wherein the multimers release monomeric IL-1ra for at least 2 days, at least 3 days, at least 4 days, at least 5 days, at least 6 days, at least 7 days, at least a week, up to 2 days, up to 3 days, up to 4 days, up to 5 days, up to days, up to 7 days, up to 8 days, up to 6±2 days in vitro.

One embodiment of the present invention provides a composition comprising multimeric IL-1ra, including IL-1raK, wherein the multimeric form comprises of insoluble multimers of IL-1ra, wherein the multimers release IL-1ra variant, such as IL-1raK, at a rate ranging from about 1 to about 7 μg/ml, at least 1 μg/ml, at least 3 μg/ml, at least 4 μg/ml, at least 5 μg/ml, at least 6 μg/ml, 1.1 to 6 μg/ml, wherein the rate of release is in the range of for about 2 days, about 3 days, about 5 days, about 7 days, about 9 days, about 10 days, at least 2 days, at least 3 days, at least 5 days, at least 7 days, up to about 10 days, at least a week, 3-10 days in vivo.

Another embodiment of the present invention provides a composition composed of multimeric IL-1ra, such as IL-1raK, wherein the multimers are non-cytotoxic, non-immunogenic, non-apoptotic and non-mitogenic in an animal or mammal, or as assessed in an animal or mammal.

The invention provides a pharmaceutical composition for the treatment or alleviation of arthritic, inflammatory, autoinflammatory, immune, autoimmune disorders in a mammalian, particularly a human subject, the composition comprising of therapeutically effective amount of multimeric IL-1ra as disclosed in the present invention. The pharmaceutical composition(s) comprises pharmaceutically acceptable carriers, additives or diluents. The composition(s) may be administered intramuscularly, intradermally, subcutaneously, intraperitoneally, inta-articularly, orally. The composition(s) may be administered through a device capable of releasing the said composition, wherein said device is selected from a group consisting of pumps, catheters, patches and implants.

In accordance with the present invention in one embodiment there is provided a multimeric form of IL-1ra, including IL-1raK, KIL-1ra and KIL-1raK, capable of treating, inhibiting and/or ameliorating inflammatory diseases or disorders, rheumatoid disease, autoinflammatory disorders or conditions resulting from adverse effects of Interleukin-1, wherein said multimers comprises non-fibrillar, aggregated and insoluble form of IL-1ra variant, such as IL-1raK, KIL-1ra, KIL-1raK, wherein said multimer(s) weakly binds to Thioflavin T and Congo-red dye. In another embodiment there is provided a multimeric form of IL-1ra, including IL-1raK, KIL-1ra and KIL-1 raK, capable of treating, inhibiting and/or ameliorating inflammatory diseases or disorders, rheumatoid disease, autoinflammatory disorders or conditions resulting from adverse effects of Interleukin-1, wherein said multimers comprises non-fibrillar, aggregated and insoluble form of IL-1ra variant, such as IL-1raK or KIL-1ra or KIL-1raK, wherein said multimers weakly binds to Thioflavin T and Congo-red dye, wherein the multimers consists of sticks of IL-1ra variant, such as IL-1raK, KIL-1ra and KIL-1raK, protein arranged together into clusters of various sizes.

In an aspect of the invention, the multimeric IL-ra or the variant IL-1ra are recombinantly produced. The present invention naturally contemplates several means for preparation of the multimeric IL-1ra, including as illustrated herein known recombinant techniques, and the invention is accordingly intended to cover such synthetic preparations within its scope. The availability of the DNA and amino acid sequences disclosed herein facilitates the reproduction of any of multimeric IL-1ra provided or contemplated herein by such recombinant techniques, and accordingly, the invention extends to expression vectors prepared from the disclosed DNA sequences for expression in host systems by recombinant DNA techniques, and to the resulting transformed hosts.

Yet another embodiment of the present invention provides a multimeric form of IL-1ra, such as IL-1 raK, KIL-1ra and KIL-1 raK, capable of treating, inhibiting and/or ameliorating inflammatory diseases or disorders, rheumatoid disease, autoinflammatory disorders or conditions resulting from adverse effects of Interleukin-1, wherein said multimers comprises non-fibrillar, aggregated and insoluble form of IL-1ra variant, such as IL-1raK, KIL-1ra and KIL-1 raK, wherein said multimers weakly bind to Thioflavin T and Congo-red dye, wherein said multimers release interleukin-1 receptor antagonist monomers at a rate of at ranging from about 1 to about 7 μg/ml, at least 1 μg/ml, at least 3 μg/ml, at least 4 μg/ml, at least 5 μg/ml, at least 6 μg/ml, 1.1 to 6 μg/ml for at least 3 days, at least 7 days, 3 to 10 days in vivo.

In an aspect of the invention is provided multimeric IL-1ra wherein said multimers weakly binds to Thioflavin T and Congo-red dye, wherein a single dose of said multimers of variants of interleukin-1 receptor antagonist ranging from 50 to 300 mg/kg body weight upon administration to a subject in need thereof reduces inflammation by at least 30%, at least 40%, at least 50%, at least 60%, up to 70%, about 40 to 70%.

In a further aspect is provided IL-1ra multimers wherein said multimers weakly bind to Thioflavin T and Congo-red dye and which multimers constitute or are a non cytotoxic, non immunogenic, non-apoptotic and non-mitogenic prodrug.

In further embodiment of the present invention there is provided a composition for treating, inhibiting and/or ameliorating inflammatory diseases or disorders, rheumatoid disease, autoinflammatory disorders or conditions resulting from adverse effects of Interleukin-1, wherein the composition comprises at least one variant of interleukin-1 receptor antagonist having amino acid sequence as set forth in SEQ ID NO: 1, SEQ ID NO: 2 or SEQ ID NO: 3.

The invention provides a pharmaceutical composition of a multimeric protein therapeutic, wherein a single dose of the composition upon administration releases said protein in an active form for a considerable period of time. The present invention provides a composition comprising multimeric IL-1ra which is stable, protease resistant and has longer shelf life than native IL-1ra.

The multimeric IL-1ra compositions of the present invention may further comprise one or more additional therapeutic agent. In an aspect thereof, the additional therapeutic agent may be an agent capable of modulating an arthritic, inflammatory or immune condition or disease. In an aspect, the therapeutic agent may be selected from a group consisting of an IL-1 specific fusion protein, anti-TNF biologicals, Etanercept, Infliximab, Humira, Adalimumab, thalidomide, a steroid, Colchicines, IL-18 BP or a derivative, an IL-18-specific fusion protein, anti-IL-18, anti-IL-18 RI, anti-IL-18 Rβ, anti-IL-1 RI, and anti IL-1 Ab.

Another embodiment of the present invention provides a process of preparation of the multimeric form of a protein peptide therapeutic, such as multimeric IL-1ra, including IL-1raK, KIL-1ra and KIL-1raK, as disclosed in the present invention, wherein the process comprises dissolving variant peptide therapeutic attached to a multimerisation motif, such as variant IL-1ra, in an embodiment IL-1raK, KIL-1ra and/or KIL-1raK, at a temperature of about 25-50° C. in a solution having pH range of about 4 to 8; and incubating the above for a period of about 6 to 48 hours with constant shaking to obtain therapeutic insoluble and aggregated multimeric form of variants of protein peptide therapeutic, such as interleukin-1 receptor antagonist. The process of preparation of the multimeric form of IL-1ra as disclosed in the present invention further comprises washing the resulting multimers with PBS; and resuspending said multimers in PBS, or such other suitable and physiologically relevant or appropriate solution. In an aspect of the process of preparation the solution may be selected from a group consisting of sodium acetate buffer having pH in the range of about 3.5 to 5.5, sodium phosphate buffer, potassium phosphate buffer and phosphate buffer (PBS) having pH in the range of 6-8 and citrate buffer in the range of 4-6. In an aspect of the process of preparation the said temperature ranges from about 30-50° C., about 30-40° C., about 32-37° C. about 37° C., at about body temperature range of temperature, preferably about 37° C., preferably 37° C.

In one embodiment there is provided a process of preparation of the composition comprising of multimeric IL-1raK, the process comprising dissolving IL-1raK at a temperature of about 25 to 50° C. in a solution having pH range of 4 to 8; and incubating the above for a period of 6 to 48 hours with constant shaking to obtain multimeric IL-1raK, wherein multimeric IL-1raK comprises insoluble multimers of IL-1raK.

The process of preparation of multimeric IL-1ra, such as IL-1raK, includes wherein the incubation period is at least 10 hours, at least 12 hours, about 12-14 hours, about 12 hours, 12 to 14 hours. In a further aspect a process of preparation of multimeric Il-1ra is provided wherein the incubation period is 6-195 hours.

In an aspect a process of preparation of multimeric IL-1ra, such as IL-1raK, is provided wherein the solution is selected from a group consisting of sodium acetate buffer having pH in the range of about 3.5 to 5.5, sodium phosphate buffer, potassium phosphate buffer and phosphate buffer (PBS) having pH in the range of 6-8 and citrate buffer in the range of 4-6.

Other objects and advantages will become apparent to those skilled in the art from a review of the following description which proceeds with reference to the following illustrative drawings.

### BRIEF DESCRIPTION OF THE DRAWINGS

FIG. 1 depicts the plasmid construct harboring the IL-1ra or IL-1raK or KIL-1ra or KIL-1raK gene.

FIG. 2 provides a line diagram showing kinetics of multimerisation at pH6.0 monitored by turbidimetric assay of IL-1ra, IL-1raK and KIL-1ra.

FIG. 3A-3C (A) Provides a bar diagram comparing changes in Thioflavin T fluorescence upon binding to amyloid fibrils formed by Aβ(1-42; positive control), multimeric IL-1raK, KIL-1ra, KIL-1raK and native IL-1raK, KIL-1ra and KIL-1raK; (B) shows a line diagram showing Congo red binding studies with native IL-1raK, KIL-1ra, multimeric IL-1raK and KIL-1ra, amyloid fibrils of Aβ(1-42; positive control); (C) shows a series of photographs showing morphologies of multimeric IL-1raK studied by AFM.

FIGS. 4A and 4B (A) shows a line diagram showing in vitro release kinetics of IL-1 raK and KIL-1ra monomers from multimeric IL-1raK and KIL-1ra formed at pH 6.0 monitored in PBS solution; (B) shows a bar diagram showing biological activity of monomers released from multimeric IL-1raK, KIL-1ra and KIL-1raK depicted as percentage inhibition of proliferation of IL-1 responsive D10 cells.

FIGS. 5A and 5B (A) shows a line diagram showing in vivo release of IL-1raK at various dosages namely 50, 100, 150, 200 and 300 mg/kg body weight; (B) provides a line diagram showing the beneficial effect of multimeric IL-1raK treatment (150 mg/kg body weight) on mean arthritic score in collagen-induced arthritis (CIA). The figure also shows the failure of non-specifically aggregated IL-1ra in treating arthritis.

FIG. 6A-6H depicts (A) a bar diagram comparing serum levels of cartilage oligomeric matrix protein (COMP) between various experimental groups; (B) a bar diagram showing serum levels of CTX II of various experimental groups; (C) a bar diagram showing serum MMP-3 levels of various experimental groups; (D) shows a series of bar diagrams comparing the levels of pro-inflammatory cytokine IL-1β; (E) shows a series of bar diagrams comparing the levels of pro-inflammatory cytokine IL-6; (F) shows a series of X-ray radiographs of representative paws from treated, untreated CIA mice and healthy mice; (G) shows a series of photographs of fore and hind limbs of one representative mouse from each experimental group. Panel A shows limbs of healthy mice; panel B multimeric IL-1raK treated mice; panel C IL-1ra treated mice; panel D disease control; and (H) shows a bar diagram showing changes in various disease parameters with treatment.

FIGS. 7A and 7B provide (A) a line diagram displaying the multimerisation kinetics of IL-1ra, GIL-1ra, IL-1raG, GIL-1raG and KIL-1ra (positive control); (B) a line diagram comparing the multimerisation profile of IL-1ra, GIL-1ra, IL-1raG and GIL-1raG.

FIG. 8 shows a line diagram displaying the multimerisation kinetics of IL-1ra, IL-1ra-KVVE, KVVE-IL-1ra, and KVVE-IL-1ra-KVVE

FIG. 9 is a line diagram displaying and comparing the multimerisation profile of KFFK-IL-1ra and EFFE-IL-1ra, IL-1ra-KFFK and IL-1ra-EFFE, KFFK-IL-1ra-KFFK and EFFE-IL-1ra-EFFE equimolar mixture and IL-1ra.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994)]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

The term “IL-1ra” refers to interleukin-1 receptor antagonist, a member of the IL-1 family that binds to the IL-1 receptor and does not induce a receptor-mediated intracellular response. IL-1ra was initially called IL-1 inhibitor and identified as a native protein in mammals (Liao Z et al (1984) J Exp Med 159(1):126-136; Liao Z et al (1985) J Immunol 134(6):3882-3886). Native (human) IL-1ra form can correspond to the sequence: RPSGRKSSKMQAFRIWDVN QKTFYLRNNQLVAGYLQGPNVNLEEKIDVVPIEPHALFLGHIGGKMCLSCVKSGDETRLQLEAVNITDL SENRKQDKRFAFIRSDSGPTTSFESAACPGWFLCTAMEADQPVSLTNMPDEGVMVTKFYFQEDE (SEQ ID NO:4). A recombinant non-glycosylated form of IL-1ra having an additional single methionine (M) residue at the N terminus (anakinra) is clinically approved (SEQ ID NO:17) for rheumatoid arthritis (RA). Non-native and variant or mutant forms of native IL-1ra, with or without an N-terminal methionine, are contemplated, particularly wherein the forms have one or more, one or a few, one to three, one to five amino acid substitutions in the sequence of native IL-1ra, or wherein the amino acid sequence has at least 80%, about 80%, at least 85%, about 85%, at least 90%, about 90%, at least 95%, about 95% amino acid sequence identity to native Il-1ra (to SEQ ID NO:4) and wherein the non-native, variant or mutant form is capable of binding to the IL-1 receptor and does not induce a receptor-mediated intracellular response.

The terms “variant IL-1ra,” “multimeric IL-1ra” “IL-1ra multimers” and any variants not specifically listed, may be used herein interchangeably, and as used throughout the present application and claims refer to proteinaceous material including single or multiple proteins having a multimerising motif or amino acid sequence attached to IL-1ra, and extends to and includes those proteins having the amino acid sequence data described herein and presented in any of SEQ ID NO: 1-3 and 5-16 or equivalent forms or variants of IL-1ra comprising the IL-1ra monomer sequence SEQ ID NO:4 and having attached one or more peptide multimerising motif, such multimerising motif having the capability of conferring or enhancing the ability of IL-1ra monomers to multimerise, and the variant or multimeric IL-1ra having profile of activities set forth herein and in the Claims. Accordingly, proteins displaying substantially equivalent or altered activity are likewise contemplated. These modifications may be deliberate, for example, such as modifications obtained through site-directed mutagenesis, or may be accidental, such as those obtained through mutations in hosts that are producers of the complex or its named subunits. Also, the terms “variant IL-1ra,” “multimeric IL-1ra” “IL-1 ra multimers” are intended to include within their scope proteins specifically recited herein as well as all substantially homologous analogs and allelic variations.

Particular exemplary multimeric IL-1ra variants are provided and described herein. These include variant IL-1ra having attached the multimerising motif KFFE, in particular having KFFE attached at the N-terminus (K-IL-1ra), at the C-terminus (IL-1raK) or at both the N- and C-termini (KIL-1raK). Thus, the term “multimeric IL-1raK” and “multimeric KIL-1 ra” used herein refers to the insoluble and protease resistant multimers of IL-1raK and multimers of KIL-1ra respectively. The term “multimeric KIL-1raK” refers to insoluble and protease resistant multimers of KIL-1raK.

A “multimerisation motif” as utilized and provided herein includes a sequence, peptide, polypeptide, chemical agent, or component, which can be attached, including covalently or recombinantly by cloning, to a peptide monomer having therapeutic capability or value or activity to enhance, facilitate or otherwise result in the multimerisation, aggregation or grouping of the monomers of the therapeutic peptide such that a relatively insoluble form of the therapeutic peptide is generated which has altered structural nature, while being capable of releasing active therapeutic monomer peptides. Thus, the multimerisation motif confers multimerising capability or capacity to a monomer peptide, while still retaining the activity of the monomer peptide on release from the multimer form.

The amino acid residues described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table of Correspondence:

TABLE OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20^{N}C below the predicted or determined Tₘ with washes of higher stringency, if desired.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

It should be appreciated that also within the scope of the present invention are DNA sequences encoding variant IL-1ra forms of the invention, which code for a variant IL-1ra having the same amino acid sequence as any of SEQ ID NOs: 1-3, 4 and 5-16, but which are degenerate to any of SEQ ID NOs: 1-3, 4 and 5-16. By “degenerate to” is meant that a different three-letter codon is used to specify a particular amino acid. It is well known in the art that the following codons can be used interchangeably to code for each specific amino acid:

| | |
|---|---|
| Phenylalanine (Phe or F) | UUU or UUC |
| Leucine (Leu or L) | UUA or UUG or CUU or CUC or CUA or |
| | CUG |
| Isoleucine (Ile or I) | AUU or AUC or AUA |
| Methionine (Met or M) | AUG |
| Valine (Val or V) | GUU or GUC of GUA or GUG |
| Serine (Ser or S) | UCU or UCC or UCA or UCG or AGU or |
| | AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG or AGA or |
| | AGG |
| Glycine (Gly or G) | GGU or GGC or GGA or GGG |
| Tryptophan (Trp or W) | UGG |
| Termination codon | UAA (ochre) or UAG (amber) or UGA |
| | (opal) |

It should be understood that the codons specified above are for RNA sequences. The corresponding codons for DNA have a T substituted for U.

Mutations can be made in the variant IL-1ra of the present invention, including SEQ ID NOs: 1-3 and 5-16, such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein.

The following is one example of various groupings of amino acids:

Amino acids with nonpolar R groups: Alanine, Valine, Leucine, Isoleucine, Proline, Phenylalanine, Tryptophan, Methionine

Amino acids with uncharged polar R groups: Glycine, Serine, Threonine, Cysteine, Tyrosine, Asparagine, Glutamine

Amino acids with charged polar R groups (negatively charged at Ph 6.0): Aspartic acid, Glutamic acid

Basic amino acids (positively charged at pH 6.0): Lysine, Arginine, Histidine (at pH 6.0)

Another grouping may be those amino acids with phenyl groups: Phenylalanine, Tryptophan, Tyrosine

Another grouping may be according to molecular weight (i.e., size of R groups):

| | |
|---|---|
| Glycine | 75 |
| Alanine | 89 |
| Serine | 105 |
| Proline | 115 |
| Valine | 117 |
| Threonine | 119 |
| Cysteine | 121 |
| Leucine | 131 |
| Isoleucine | 131 |
| Asparagine | 132 |
| Aspartic acid | 133 |
| Glutamine | 146 |
| Lysine | 146 |
| Glutamic acid | 147 |
| Methionine | 149 |
| Histidine (at pH 6.0) | 155 |
| Phenylalanine | 165 |
| Arginine | 174 |
| Tyrosine | 181 |
| Tryptophan | 204 |

Particularly preferred substitutions are:

Lys for Arg and vice versa such that a positive charge may be maintained;
Glu for Asp and vice versa such that a negative charge may be maintained;
Ser for Thr such that a free —OH can be maintained; and
Gln for Asn such that a free NH₂ can be maintained.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces -turns in the protein's structure.

Two amino acid sequences are “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

As used herein, “pg” means picogram, “ng” means nanogram, “ug” or “μg” mean microgram, “mg” means milligram, “ul” or “μl” mean microliter, “ml” means milliliter, “l” means liter.

The phrase “pharmaceutically acceptable” refers to molecular entities and compositions that are physiologically tolerable and do not typically produce an allergic or similar untoward reaction, such as gastric upset, dizziness and the like, when administered to a human.

The term “therapeutically effective amount” as used herein refers to an amount of a therapeutic agent to treat, ameliorate, or prevent a desired disease or condition, or to exhibit a detectable therapeutic or preventive effect. The precise effective amount for a subject will depend upon the subject's size and health, nature and extent of condition, and the therapeutics or combination of therapeutics selected for administration. The effective amount for a given situation is determined by routine experimentation and is within the judgment of the clinician.

The term “inhibit” used herein means to reduce (wholly or partially) or to prevent.

Protein, polypeptides or other compounds described herein are expressed, purified or isolated. A purified or isolated composition (e.g., protein, polypeptide) is at least 60% by weight (dry weight) the compound of interest. Preferably, the preparation is at least 75%, more preferably at least 90%, and most preferably at least 99%, by weight the compound of interest. Purity is measured by any appropriate standard method, for example, column chromatography, polyacrylamide gel electrophoresis, or HPLC analysis. The protein or polypeptide is purified from MSC culture media or recombinantly produced.

In a general aspect, the present invention provides novel and useful forms of IL-1ra which provide sustained release of active monomers of IL-1ra and long-acting forms of IL-1ra. Native and even recombinant IL-1ra (as commercialized in the form of anakinra) is recognized to possess only a 4-6 hour half life in serum in patients, particularly humans. The novel and useful forms of IL-1ra herein provided are multimeric and release active monomers of IL-1ra in a sustained manner. The multimeric IL-1ra forms provide long-acting, useful and effective IL-1ra capable of inhibiting, treating and/or ameliorating rheumatoid disease, acute and chronic inflammatory diseases or disorders, auto inflammatory disorders or conditions resulting from adverse effects of Interleukin-1 (IL-1), including rheumatoid arthritis (RA), Inflammatory Bowel Disease (IBD), Ulcerative colitis (UC), and acute hepatic injury.

Aggregation is a dominant degradation pathway of proteins and can occur during all stages of protein therapeutics and storage (Cleland, J. F et al (1993) Crit. Rev The. Drug Carrier Syst. 10:307-377; Carpenter, J. F et al (1999) Methods Enzymol 309:236-255; Fink, A. L. (1998) Protein Fold Des 3:R9-R23; Manning, M. C et al (1989) Pharm Res 6:903-918). The aggregation of proteins and their deposition into amorphous precipitates or insoluble fibrils is also linked to a number of amyloid diseases, such as Alzheimer's and Parkinson's disorders (Koo, E. H. et al (1999) Proc Natl Acad Sci USA 96:9989-9990; Hardy, J., and D. J. Selkoe. (2002) Science 297:353-356; Kyle, R. A. (1994) Ann RevMed 45:71-77). The aggregation of proteins is often a problem resulting in relatively useless or problematic contaminants which may pose problems in safety, efficacy, and immunogenicity of protein therapeutics in vivo, as well as problems to be avoided in formulation strategies.

IL-1ra has been reported to form aggregates at high concentrations (e.g. 100 mg/ml and above), at high temperature (e.g. 39-48° C.) or at high pressure (e.g. hydrostatic pressure>180 MPa) (Krishnan S et al (2009) Biophys J 96(1):199-208; Seefeldt M B et al (2005) Prot Sci 14(9):2258-2266). The non-specific aggregation reported by Krishnan et al results in structural perturbations in the form of a transition from intramolecular beta sheet formation to inter-molecular beta sheet formation. IL-1ra is particularly sensitive to pressure, having an unfolding transition that begins at 140 MPa, and relatively low pressure (˜200 MPa) causes IL-1ra to aggregate. The elevated pressures increase the population of IL-1ra denatured conformations and enables aggregation through intermolecular non-native disulfide crosslinking (Seefeldt M B et al (2005) Prot Sci 14(9):2258-2266). Chang et al earlier reported that at conditions near room temperature (30° C. and atmospheric pressure) IL-1ra forms intramolecular disulfide bonds which result in slight structural modification and irreversible soluble dimerization (Chang B S et al (1996) Biophys J 71:3399-3406). These dimers retain about two-thirds of the activity of the native monomer and are noted as problematic degradation products and aggregates formed during long term storage of the recombinant IL-1ra product.

Thus, protein aggregates and multimers have historically been viewed as problematic contaminants and at least relatively inactive if not disease-associated protein forms. In the present invention, however, active and particularly useful multimeric forms of the IL-1ra protein therapeutic are now provided. These multimeric IL-1ra forms release active IL-1ra monomers and provide a sustained release depot of active protein monomers for therapeutic applications.

Recently, active aggregated and insoluble supramolecular assembly forms of insulin have been described, which release active insulin monomers in vitro and in vivo (Gupta S et al (2010) PNAS USA 107(30):13246-13251). These active insulin aggregated oligomers are further described in published patents US2009/0258818 and WO2009/125423, incorporated herein by reference. In the case of insulin, these active insoluble forms were generated naturally in solution using particular preparation method and conditions, without requiring alteration or addition to the native insulin monomer sequence or solution.

In the case of certain protein therapeutic monomers, for instance IL-1ra, however, and in contrast, the native monomer does not readily form useful or active aggregates or multimers under standard conditions. As described herein, native IL-1ra showed minimal changes in multimerisation profile during incubation assessments. Incubation included 37° C. incubation at pH approximately 6 in phosphate buffer (50 mM), agitated at 180 rpm for up to 8-10 hours. Multimerisation was monitored via turbidity, assessing OD at 405 nM. Aggregated IL-1ra formed under aggregation promoting conditions as above and previously described and reported, such as high concentration, high temperature, and high pressure, are not useful or particularly active molecules or forms. The present examples describe inactivity of aggregated IL-1ra, including in arthritis model systems. The aggregated IL-1ra may be prepared by a process comprises dissolving IL-1 receptor antagonist (IL-1ra) in buffer at about pH 6 and incubating at elevated temperature, in one such embodiment incubation in 10 mM sodium citrate, 140 mM NaCl, and 0.5 mM EDTA, pH 6.5 (CSE) buffer and incubating at 47° C. for 2-4 hours.

In accordance with the present invention, attachment of one or more multimerising motif to the IL-1ra molecule or peptide confers multimerisation capability to the IL-1ra molecule, such that the IL-1ra multimerises to a form of IL-1ra which acts as a reservoir for sustained release of active IL-1ra monomers. The present inventors have discovered that IL-1ra variants which possess or incorporate multimerising or multimerisation promoting sequence(s) or motif(s), including by covalent attachment, for instance at the N-terminus, at the C-terminus, or at both the N- and C-terminus of the native IL-1ra sequence, form active sand useful multimeric forms which release active IL-1ra monomers sustainably and over a long term/extended time period to provide long acting extended half-life forms of IL-1ra. The released IL-1ra monomers are active in vitro and in vivo, including in collagen-induced arthritis animal models.

The IL-1ra multimers are distinct in several aspects versus IL-1ra monomers and also versus aggregates of IL-1ra (e.g., aggregates formed under high temperature, high pressure, high concentration, low pH or such other amyloidogenic conditions). The IL-1ra multimers of the present invention possess one or more of the following characteristics: they demonstrate protease resistance; demonstrate relatively weak binding to Congo Red, particularly as compared to amyloid Aβ; demonstrate a relatively low increase in thioflavin-T fluorescence, showing on the order of about 3 fold increase versus IL-1ra monomers, whereas Aβ amyloid shows at least about 100 fold increase in thioflavin-T fluorescence; the multimers provide a protein depot for release of IL-1ra monomers at a site of injection in an animal or mammal; the multimers release IL-1ra into circulation after injection in an animal or mammal; the multimers release IL-1ra for many hours, even many days, at least 1 day, at least 2 days, at least 3 days, at least 5 days, at least 7 days, depending on the amount of multimer infused or injected, versus IL-1ra which has a half life of approximately 4-6 hours on infusion of 100 mg.

In accordance with the present invention, one or more multimerisation promoting, facilitating, or enhancing motif, including a peptide, peptide-like, chemical, biological sequence or agent is attached or otherwise directly associated with the or to the active protein therapeutic monomer, for instance IL-1ra, so as to promote the multimerisation of the monomer, such as IL-1ra, to form a multimer which is capable of sustainably releasing active monomer, such as IL-1ra monomer(s), in vitro and in vivo. The multimerising motif(s) act to promote productive association of protein monomers to form multimers which retain the ability to release active monomers over sustained lengths of time, thereby providing a depot of monomers. Thus, these variant monomers with multimerising motifs provide enhanced half-life or long-acting protein therapeutics.

The multimerising motif may be selected from any sequence, peptide, or attachable agent or compound with capability for promoting multimerisation of a monomer. For example, in the study of the formation of toxic oligomers and fibrillar aggregates such as the Aβ peptide implicated in Alzheimer's disease, it has been recognized that amyloid fibril assembly is based to a certain, if not large, extent on fundamental properties of the polypeptide chain. Fragments of the Aβ peptide as well as synthetic peptides with de novo sequences have been shown to form amyloid in vitro (Zhang, S. (2002) Biotechnol. Adv. 20:321-339; Aggeli, A. et al (2001) Proc Natl Acad Sci USA 98:11857-11862; Lu, K. et al (2003) J Am Chem So 125:6391-6393). Tjerenberg et al showed that synthetic peptides as short as four residues can self-assemble and form amyloids in vitro (Tjernberg, L. et al (2002) J Biol Chem 277:43243-43246). In Tjerenberg's study, the peptide with the highest fibrillation propensity was KFFE. This peptide is now demonstrated in the present invention to confer positive and useful multimerisation capability to a peptide monomer, as exemplified herein in IL-1ra, capable of generating active multimers with sustained release and long acting therapeutic capability and activity. Many naturally existing and synthetic peptides that aggregate to form fibrils contain aromatic residues, including the KFFE peptide, the 16-22 fragment of Aβ(KLVFFAE (SEQ ID NO:30)), the NGAIL fragment of amylin, islet amyloid polypeptides NFLV and FLVHS, the peptide NFGSVQFV, the peptide GNNQQNY and various fragments of calcitonin, including DFNKF and DFNK (Lu, K. et al (2003) J Am Chem Soc 125:6391-6393; Azriel, R., and E. Gazit (2001) J Biol Chem 276:34156-34161; Mazor Y et al (2002) J Mol Biol 322:1013; Haggqvist B et al (1999) PNAS USA 96:8669-8674; Balbirnie M et al (2001) PNAS USA 98:2375-2380; Reches, M. et al (2002) J Biol Chem 277:35475-35480). The tripeptides Boc-Ala-Aib-Val-OMe, Boc-Ala-Aib-Ile-Ome and Boc-Ala-Gly-Val-OMe have been shown to form supermolecular beta sheet structures and aggregate into amyloid-like fibrils (Maji S K et al (204) Tetrahedron 60:3251). The simple aromatic Phe-Phe dipeptide also has been shown to promote self-assembly (Song Y J et al (2004) Chem Commun 9:1044). The microcin E492 peptide, an 84 amino acid mature peptide naturally produced by Klebsiella pneumonia assembles in vitro into amyloid-like fibrils, and amyloid formation in vivo is associated with loss of bacterial toxicity of the protein (Bieler S et al (2005) J Biol Chem 280(29):26880-26885; Genbank AAD04332.2). The transthyretin (TTR) protein is well-recognized as one of several proteins known to cause amyloid disease, including in humans, and has homologs in many diverse species with varying degrees of amino acid sequence similarity (Lundberg E et al (2009) FEBS J 276:1999-2011). Ehud Gazit has undertaken an extensive study of self assembly of short aromatic peptides into amyloid fibrils and related nanostructures and describes numerous peptide sequence candidates for multimerisation motifs (Gazit E (2007) Prion 1(1):32-35; Gazit E (2002) The FASEB J 16:77-83; Gazit E (2005) FEBS J 272:5971-5978). Naturally occurring oligomerization modules include the coiled coil leucine zippers, such as the GCN4 leucine zipper (Landschulz W H et al (1988) Science 240:1759-1764; O'Shea E K et al (1991) Science 254:539-544). The GCN4 leucine zipper core sequence is RMKQLEDKVEELLSKKYHLENEVARLKKLVGER (SEQ ID NO:31) (RSCB Protein Data Bank, rscb.org/pdb). Zhang et al have reported a genetic selection scheme to search libraries for peptides that are able to mediate homodimerization or higher-order self-oligomerisation of a protein in vivo (Zhang Z et al (1999) Current Biology 9:417-420).

As provided in the instant application, various exemplary and candidate mulitmerisation sequences have been attached via recombinant means as described herein to monomeric IL-1ra. After cloning and expression of any of the variant IL-1ra sequences with attached one or more candidate multimerisation domains, the variant IL-1ra is tested and monitored for multimerisation, including using turbidimetric assays, and for active monomer release and monomer activity, including as exemplified and described herein. The Examples herein describe assessment of numerous exemplary candidate multimerisation motifs on a protein therapeutic monomer, such as IL-1ra, and including motifs KFFE, KVVE, KFFK, EFFE and GNNQQNNY. The Examples particularly describe the generation of useful multimers of variant IL-1ra having one or more attached KFFE multimerisation motif, the variant IL-1ra KFFE multimers (denoted herein IL-1raK, K-IL-1ra and K-IL-1raK) are capable of releasing active monomers over extended periods of time, particularly over days, in vitro and in vivo. In addition, multimeric IL-1raK is demonstrated to be affective for treatment and amelioration of arthritis, colitis and induced liver injury in animal model systems, and in each instance multimeric-IL-1ra was more effective than monomeric IL-1ra in these models. The in vivo effect of the composition comprising, for example multimeric IL-1raK, on controlling various serum parameters of inflammation and cartilage damage such as proinflammatory cytokines (IL-1, IL-6), cartilage oligomeric matrix protein (COMP), matrix metalloproteinase-3 (MMP-3), etc, have been verified using collagen induced arthritic mice.

Thus, multimeric IL-1ra, which corresponds to IL-1ra attached to a multimerisation motif and aggregated as multimer(s), provides a useful and applicable IL-1ra therapeutic composition for alleviation and treatment of IL-1 mediated disorders, conditions or diseases, including arthritic, auto-immune and inflammatory diseases and conditions.

In an aspect of the invention, the multimers of the present invention may further incorporate or include additional attachments, including useful or applicable peptides, agents, compounds, sequences, targets, receptors, ligands, and/or toxins. These additional attachments may serve in one or more uses or applications such as: in labeling the multimer(s); in providing enhanced stability or protease resistance; in targeting the multimer(s) for action/activity at a particular location, cell or tissue type; in targeting the multimer(s) to a receptor or ligand of choice or preference, such as a cell surface receptor; as a target or receptor for directed killing of a cell or even for destruction of the multimer(s) by proteolytic, enzymatic or other directed attack such as to eliminate or degrade the remaining multimer(s) after injection or administration for a set or desired period of time. The multimers may further incorporate additional drugs or agents, useful in therapy or amelioration of arthritic, auto-immune or inflammatory conditions, such as other DMARDs, or chemical agents such as immune modulators, or anti-inflammatory agents.

The invention provides compositions of multimeric therapeutic peptides, including as exemplified herein multimeric IL-1ra. The compositions may be therapeutic compositions or pharmaceutical compositions, formulated or suitable for administration to an animal, including a mammal, particularly a human. As will be appreciated by those in the art, a variety of solutions such as known buffers can be used for preparation, re-suspension, storage and washing of the multimeric IL-1ra disclosed in the present invention.

A pharmaceutical composition may comprise one or more multimeric therapeutic peptide of the present invention and may also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Typically, the therapeutic compositions are prepared as injectables, either as liquid solutions or suspensions; solid forms suitable for solution in, or suspension in, liquid vehicles prior to injection can also be prepared. Liposomes and neosomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

The present invention further contemplates therapeutic compositions useful in practicing the therapeutic methods of this invention. A subject therapeutic composition includes, in admixture, a pharmaceutically acceptable excipient (carrier) and one or more of a multimeric therapeutic polypeptide, an analog thereof or fragment thereof, as described herein as an active ingredient. In a preferred embodiment, the composition comprises a multimeric IL-1ra capable of modulating the IL-1 receptor or modulating IL-1 receptor ligand binding or activity.

The preparation of therapeutic compositions which contain polypeptides, analogs or active fragments as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A polypeptide, analog or active fragment can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic polypeptide-, analog- or active fragment-containing compositions are conventionally administered intravenously or subcutaneously, as by injection of a unit dose, for example. A variety of administrative techniques may be utilized, among them parenteral techniques such as subcutaneous, intravenous and intraperitoneal injections, catheterizations and the like. Average quantities of the multimers may vary and in particular should be based upon the recommendations and prescription of a qualified physician or veterinarian. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of inhibition or neutralization of IL-1 capacity desired. Precise amounts of active ingredient required to be administered depend on the judgment of the practitioner and are peculiar to each individual. Anakinra (Kineret™) recombinant IL-1 ra presently in clinical use is administered at approximately 1-2 mg/kg dosing, with a daily subcutaneous injection of approximately 100 mg/day. The IL-1 ra multimers of the present invention may be clinically utilized by administering a higher dose less frequently. Thus, suitable dosages for the present IL-1 ra multimers may be at higher doses than for wild type IL-1ra and with less frequent administration, in as much at the present IL-1ra multimers act as depots of Il-1ra and release monomers over time. One of skill in the art may extrapolate suitable dosing in another mammal based on the doses provided and shown herein in mice. For a larger animal, larger doses equally or less frequently may be suitable, or equivalent doses more frequently may be suitable, for example. In any event, the dosing in humans for the IL-1ra multimer will be a larger dose than anakinra administered less frequently than anakinra, in as much as the IL-1ra multimer(s) of the invention provide a longer acting and sustained release form of Il-1ra. Dosing in humans, for example, may range from about 2×, 3×, 5×, 7×, 10×, 15×, 20×, 25×, 30×, 40×, 50×, up to 100× the normal dose of wild type, including anakinra, Il-1ra. Administration may be every 3 days, every week, every 2 weeks, every 3 weeks, every month, every 2 months, every 3 months, etc as appropriate based on the amount of multimer administered in each dose and the monomer release kinetics. Suitable regimes for initial administration and additional subsequent administration, or for repeated and/or regular administration, are also variable, but are typified by an initial administration followed by repeated doses at one or more day, week, even month intervals by a subsequent injection or other administration.

The therapeutic compositions may further include an effective amount of the multimer or analog thereof, and one or more of the following active ingredients: a disease-modifying anti-rheumatic drug (DMARD), an anti-inflammatory agent, an immune modulator, a cell proliferation modulator or anti-mitotic, a pain medication or analgesic, an antibiotic, a steroid.

The compositions provided in the present invention comprise multimeric protein forms of one or more relevant/applicable therapeutic protein(s) and are applicable for treatment or amelioration of a number of chronic diseases and acute symptoms in mammals, in particular, human subjects. The compositions disclosed in the present invention comprises multimers of therapeutic proteins particularly the multimeric form of the protein for sustained release of the protein.

In accordance with the invention, biopharmaceuticals, particularly therapeutic peptides, can be induced to multimerise by incorporating one or more of a multimerising motif(s). Compared to the native form of the soluble proteins, these supra-amorphous multimers gain new properties such as enhanced stability, protease resistance, longer shelf life and can serve as a concentrated compact source of molecules.

The multimeric forms of the Il-1ra peptide of the invention, including IL-1 raK and KIL-1ra disclosed in the present invention, exist with minimal structural perturbations. Any such perturbations do not serve to alter the inherent IL-1 receptor binding and inhibiting activity of the released monomeric IL-1ra. Multimerisation results in a change in the solubility of IL-1raK and KIL-1ra molecules. Significantly, release of IL-1raK and KIL-1ra monomers from their respective multimeric forms is biologically active and thus resembles the native IL-1ra structure.

The present invention provides a composition comprising of multimeric IL-1raK capable of sustained release of IL-1raK monomers. The composition comprising IL-1 raK multimers is useful in down-modulating the adverse effects of IL-1.

The present invention provides a composition comprising multimers of IL-1ra, for example in the form of multimeric IL-1raK, that is useful in combating and controlling the undesirable inflammatory responses mediated by interleukin-1 (IL-1) in mammals, in particular, human subjects. The multimeric IL-1raK when injected subcutaneously controls the inflammatory responses for prolonged periods even when the plasma levels are not detectable, thus affording a long lasting treatment of inflammatory disorders such as arthritis in human subjects suffering from the above mentioned condition.

According to the present invention, one embodiment provides a composition comprising multimeric IL-1raK that causes a sustained release of IL-1raK monomers over a period of days, as opposed to hours. Active monomers are, in an aspect of the invention, released in amounts of at least 1 μg/ml, in particular ranging between 1-6 μg/ml, and lasting for a period of days, at least 1 day, and particularly at least 3 days, particularly about at least 3-5 days, when administered subcutaneously.

For purposes of the present invention, an effective dose of the composition comprising multimeric IL-1ra, including particularly IL-1raK, will generally be from about 50 mg/kg to about 100 mg/kg or about 100 mg/kg to about 200 mg/kg, or about 100 mg/kg to about 300 mg/kg, or about 100 mg/kg, or about 150 mg/kg, or about 200 mg/kg of the compositions of the present invention in the subject to which it is administered. In an aspect hereof, the dosage of the composition comprising multimeric IL-1raK wherein the dosage ranging from about 50 mg/kg to about 300 mg/kg body weight was monitored for the experimental period.

The composition of multimeric IL-1ra, particularly including IL-raK, is stable, protease resistant and has longer shelf life than monomeric IL-1ra, ranging from about 3 to 10 days, at least 3 days, at least 5 days, at least 7 days, at least 10 days, or more. In yet another embodiment if the present invention there is provided composition comprising multimeric IL-1raK which capable of releasing IL-1raK monomers in a controlled manner for a substantial period of time in mammals, in particular, human subjects. The composition comprising the multimeric IL-1raK is capable of releasing IL-1raK monomers at constant rate both in vitro and in vivo. Further, a composition comprising the multimeric IL-1ra, including IL-1raK of the present invention, can be used as a single dose for long lasting effects that frees the patients from the need to administer IL-1ra daily.

In yet another embodiment of the present invention, zero order kinetics or sustained release is observed for the in vivo release of IL-1raK monomers from multimeric IL-1raK. The IL-1ra released from multimeric variant IL-1ra is equivalent in biological function to soluble IL-1ra.

The invention provides methods for the effective and long lasting treatment of disorders or diseases related to or caused by adverse effects of interleukin-1 in mammals, in particular, human subjects, including arthritic disease, inflammatory conditions, and auto-immune disorders. The method(s) of the invention include the effective and long lasting treatment of rheumatoid arthritis and ulcerative colitis in human subjects. These methods utilize or incorporate the administration of one or more multimeric form of IL-1ra in limited doses. Thus, instead of daily dosing as with monomers of IL-1ra. In accordance with the invention, the method includes a single dosing or infusion of multimeric IL-1ra for sustained release of IL-1ra monomer(s) over at least one day or days.

In an aspect of the present invention, composition comprising IL-1ra multimers is capable of decreasing the number of Th17 cells in treated animals. In yet another embodiment of the present invention, the composition comprising IL-1raK is capable of increasing the number of regulatory T-cells in the lymphoid organs of the treated animal.

In still yet another embodiment of the present invention, composition comprising IL-1ra multimers, as exemplified by IL-1raK, is capable of arresting and slowing the radiographic progression of joint damage in subjects suffering from arthritis. Arthritic animals treated with the composition comprising multimeric IL-1raK showed a significant reduction, on the order of a ˜50% reduction, particularly at least 70% reduction in clinical signs and symptoms of the disease. In assessing reduction of clinical signs and symptoms of disease, one skilled in the art may measure the levels of various serum parameters such a cartilage oligomeric matrix protein (COMP), CTX II, matrix metalloproteinase-3 (MMP-3), proinflammatory cytokines (IL-1β, IL-6), to demonstrate the effectiveness of treatment.

In accordance with the methods, the composition comprising multimeric IL-1ra is capable of reducing the disease activity index in animals with inflammatory disease, as exemplified herein by experimental colitis. The composition comprising multimeric IL-1ra, including multimeric IL-1raK, is capable of reducing the serum levels of hepatic enzymes, ALT and AST, which are markers of hepatic injury and inflammation in an animal or mammal, including a human. The composition comprising multimeric IL-1ra, particularly IL-1raK, is capable of reducing the severity of drug induced liver toxicity and inflammation.

The current methodology can be extended to those chronic and inflammatory diseases in mammals, in particular, human subjects, where a sustained and continuous therapy is required using peptides, proteins, or small molecules.

The multimeric forms of variants of interleukin-1 receptor antagonist may be glycosylated or non-glycosylated and can be expressed in a prokaryotic expression system for example E. coli cell or a eukaryotic expression system for example mammalian cell.

The multimeric forms of IL-1raK, KIL-1ra and KIL-1raK as disclosed in the present invention release natively folded and biologically active monomers or molecules and are capable of binding IL-1 receptor type I and II and blocking IL-1 signaling pathway.

The multimers as disclosed in the present invention are capable of reducing the activation and proliferation of autoinflammatory Th17 cells in various lymphoid organs of the treated animals as assessed by flow cytometry (FACS) and cytokine ELISA. It was observed that the multimers upon administration increase the number of regulatory T-cells in the treated animals as measured by flow cytometry and cytokine ELISA. The numbers of activated Th17 cells are assessed by lineage specific markers and cytokines such as RORγt, IL-17R, IL-23, IL-17 and the number of regulatory T-cells are quantified using cell-specific markers such as FOXP-3, CD25, Glucocorticoid-induced Tumor necrosis factor receptor family-Related (GITR), Cytotoxic T-Lymphocyte Antigen-4 (CTLA-4).

Further, it was also observed that the in vivo release of monomers from the multimers is capable of reducing disease severity in an animal model of inflammation, wherein the inflammation scoring system consists of scoring the extent of redness and swelling by macroscopic observation of the joints of hind and forelimbs of the experimental animals and measuring the changes in individual paw volumes using a plethysmometer.

Surprisingly it was observed that a single injection of the multimeric forms of IL-1raK, KIL-1ra and KIL-1raK as disclosed in the present invention into the diseased animal reduces the paw inflammation as assessed by subjective scoring and plethysmometer by 40-60%. Further, the treated animals showed a 70% reduction in histological scoring of the knee joint as revealed by a reduction or absence of inflammation, destruction of articular cartilage, bone erosion or proteoglycan depletion. The radiographic scoring revealed a 70-80% reduction in bone destruction in treated animals in comparison to disease controls and aggregated IL-1ra and correlated well with the histological scores. The radiographs of the diseased animals showed severe destructive abnormality with all the metatarsal bones and severe bone erosion in most of the tarsometatarsal, metatarsophalangeal and knee joints in comparison to the treated animals. The treatment of the diseased animals with the multimers increases their physical and mechanical (motor) ability as assessed by grip strength analysis and video-taping of movements.

The frequency of administration of the pharmaceutical composition comprising the multimeric forms of IL-1ra, particularly IL-1raK, KIL-1ra and KIL-1raK as disclosed in the present invention, may be every several days, weekly or biweekly or monthly for significant, complete or near complete remission or extended (e.g. long term) reduction of the symptoms or condition being treated.

Methods for preparation of multimeric IL-1ra are provided herein. Multimeric motifs are attached to the protein therapeutic monomer by chemical attachment, or by cloning and recombinant expression of a fusion IL-1ra. The variant IL-1ra with mutlimerisation motif(s) are incubated in solution to generate multimers of IL-1ra. The multimers of IL-1ra may be prepared in solution at a pH ranging from about 4 to about 8, particularly from pH 4 to pH8, particularly about pH 6, particularly pH 6-7, particularly pH 6.

Also, antibodies including both polyclonal and monoclonal antibodies, may possess certain diagnostic applications and may for example, be utilized for the purpose of detecting and/or measuring conditions such as the extent or severity of an arthritic condition or IL-1 mediated disease, the amount of IL-1, or the like. Antibodies may be utilize to detect and evaluate the amount of multimeric IL-1ra in an individual or patient following infusion or to monitor the amount of the depot multimer remaining or its location(s). For example, the multimeric IL-1ra or its subunits may be used to produce both polyclonal and monoclonal antibodies to themselves in a variety of cellular media, by known techniques such as the hybridoma technique utilizing, for example, fused mouse spleen lymphocytes and myeloma cells. Likewise, small molecules that mimic or antagonize the activity(ies) of the multimer of the invention may be discovered or synthesized, and may be used in diagnostic and/or therapeutic protocols.

The general methodology for making monoclonal antibodies by hybridomas is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890.

Panels of monoclonal antibodies produced against peptides can be screened for various properties; i.e., isotype, epitope, affinity, etc. Of particular interest are monoclonal antibodies that neutralize the activity of the multimer or of IL-1. Such monoclonals can be readily identified in IL-1 or IL-1ra activity assays. High affinity antibodies are also useful when immunoaffinity purification of native or recombinant IL-1ra is possible.

Preferably, the anti-multimer antibody used in the diagnostic methods of this invention is an affinity purified polyclonal antibody. More preferably, the antibody is a monoclonal antibody (mAb). In addition, it is preferable for the anti-multimer antibody molecules used herein be in the form of Fab, Fab′, F(ab′)₂ or F(v) portions of whole antibody molecules.

Methods for producing polyclonal anti-polypeptide antibodies are well-known in the art. See U.S. Pat. No. 4,493,795 to Nestor et al. A monoclonal antibody, typically containing Fab and/or F(ab′)₂ portions of useful antibody molecules, can be prepared using the hybridoma technology described in Antibodies—A Laboratory Manual, Harlow and Lane, eds., Cold Spring Harbor Laboratory, New York (1988), which is incorporated herein by reference. Briefly, to form the hybridoma from which the monoclonal antibody composition is produced, a myeloma or other self-perpetuating cell line is fused with lymphocytes obtained from the spleen of a mammal hyperimmunized with a multimer, a binding portion thereof, or IL-1ra.

Splenocytes are typically fused with myeloma cells using polyethylene glycol (PEG) 6000. Fused hybrids are selected by their sensitivity to HAT. Hybridomas producing a monoclonal antibody useful in practicing this invention are identified by their ability to immunoreact with the multimer and their ability to inhibit specified multimer activity or alter IL-1 or IL-1 receptor activity in target or relevant cells.

A monoclonal antibody useful in practicing the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques.

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host.

Such operative linking of a DNA sequence of this invention to an expression control sequence, of course, includes, if not already part of the DNA sequence, the provision of an initiation codon, ATG, in the correct reading frame upstream of the DNA sequence.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMB9 and their derivatives, plasmids such as RP4; phage DNAS, e.g., the numerous derivatives of phageλ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products.

Considering these and other factors a person skilled in the art will be able to construct a variety of vector/expression control sequence/host combinations that will express the DNA sequences of this invention on fermentation or in large scale animal culture.

Synthetic DNA sequences allow convenient construction of genes which will express IL-1ra analogs or “muteins” or IL-1ra multimers having multimerisation motifs. Alternatively, DNA encoding muteins can be made by site-directed mutagenesis of native IL-1ra genes or cDNAs, and muteins can be made directly using conventional polypeptide synthesis.

A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). This method may be used to create analogs with unnatural amino acids.

Labels may be employed in the multimers or multimer constructs including radioactive elements, enzymes, chemicals which fluoresce when exposed to ultraviolet light, and others. A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate. The multimer(s) or its binding partner(s) can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

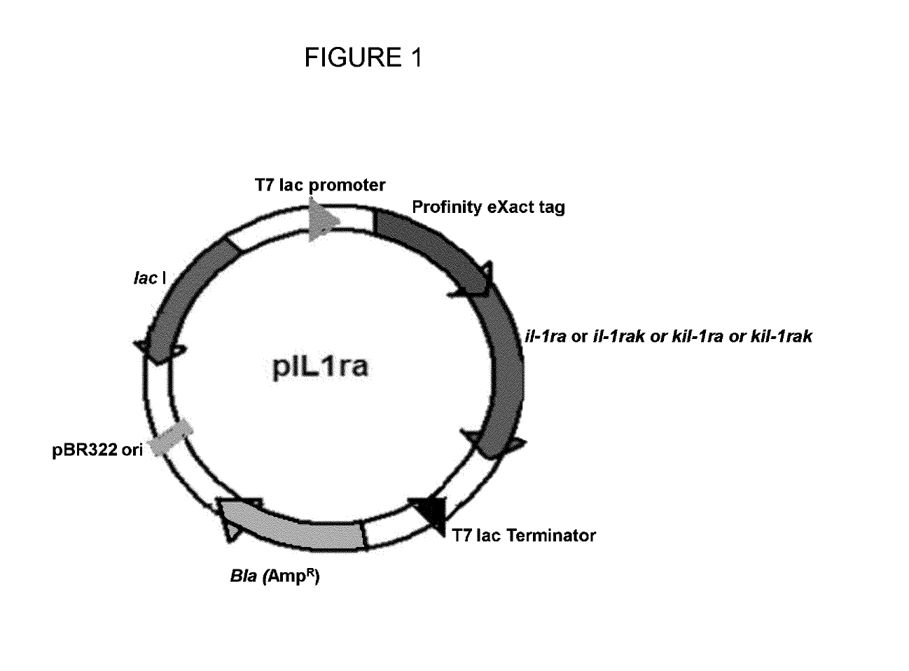

The construction, expression and assessment of various IL-1ra multimers is described herein, including in the Examples provided. Example 1 describes cloning, expression and purification of IL-1ra, IL-1raK, KIL-1ra and KIL-1raK. The resulting cDNA with additional residues corresponding to the multimerising motif KFFE and the N-terminal purification tag were cloned into pPAL7. FIG. 1 shows the plasmid construct of IL-1ra, IL-1raK, KIL-1ra and KIL-1raK genes. The recombinant IL-1ra, IL-1raK, KIL-1ra and KIL-1raK were expressed in BL21 cells and their identity was confirmed by N-terminal sequencing and western blotting using anti-IL-1ra antibody.

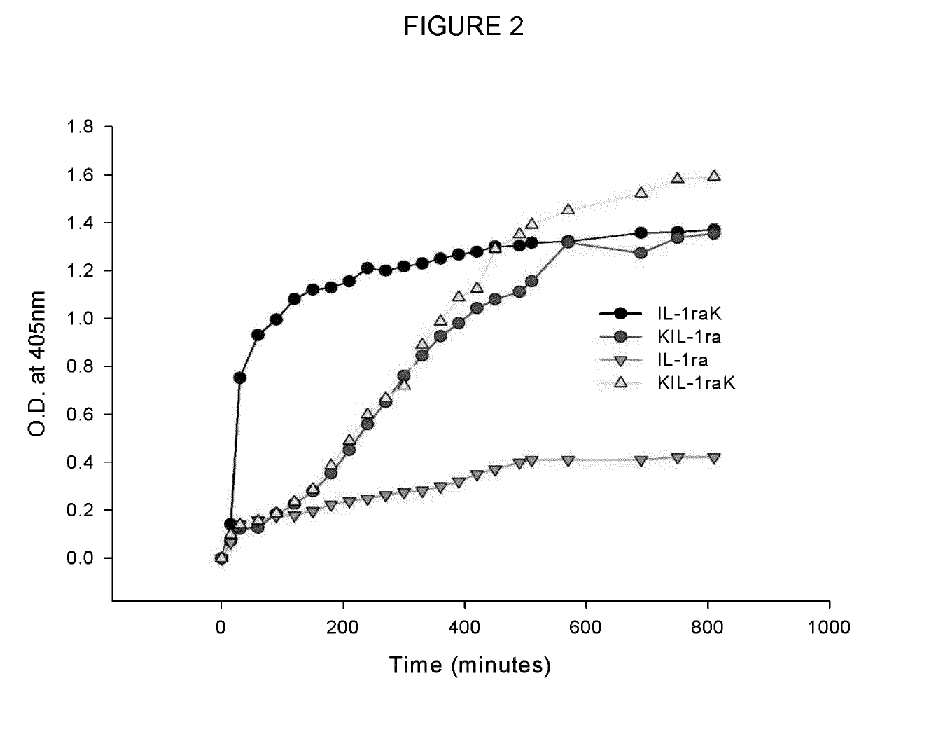

Example 2 describes formation of multimers of IL-1raK, KIL-1ra and KIL-1raK. Multimerisation was monitored using turbidimetric assay. IL-1ra isoform with C-terminal KFFE (IL-1raK) showed fast and sudden multimerisation kinetics in comparison to IL-1ra with N-terminal KFFE (KIL-1ra) and KIL-1raK i.e. IL-1ra with KFFE at both N and C-termini when agitated at 180 rpm at 37° C. at pH 6.0 in 50 mM phosphate buffer, which became saturated at around 8-10 hours of incubation. The multimerisation profile of IL-1ra on the other hand showed minimal changes during the observed incubation period indicating the contribution of multimerising motif in aiding the multimerisation process. FIG. 2 shows the kinetics of multimerisation of IL-1ra variants.

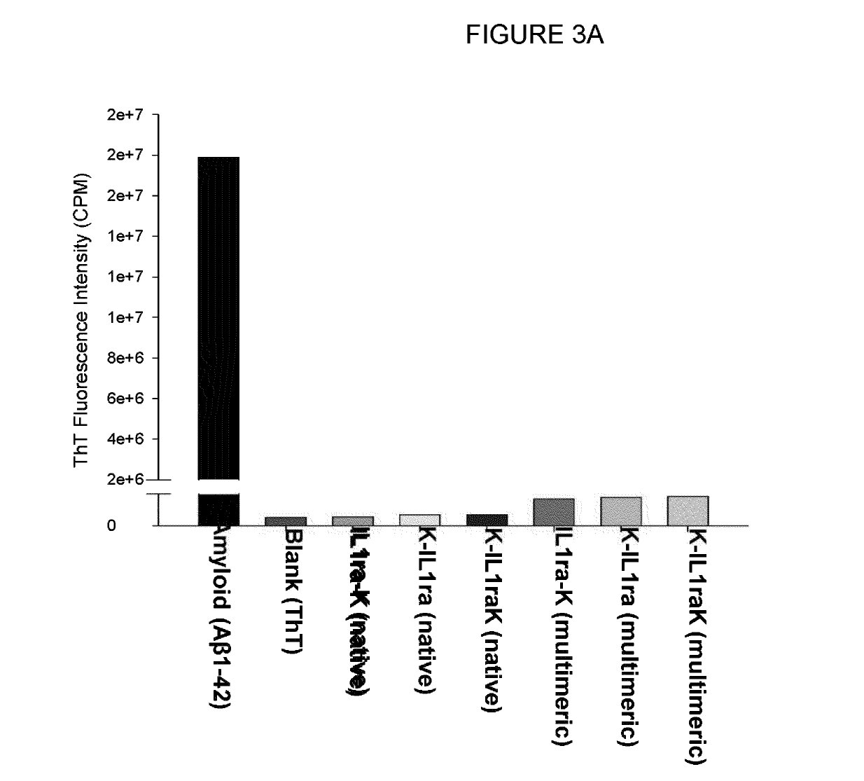

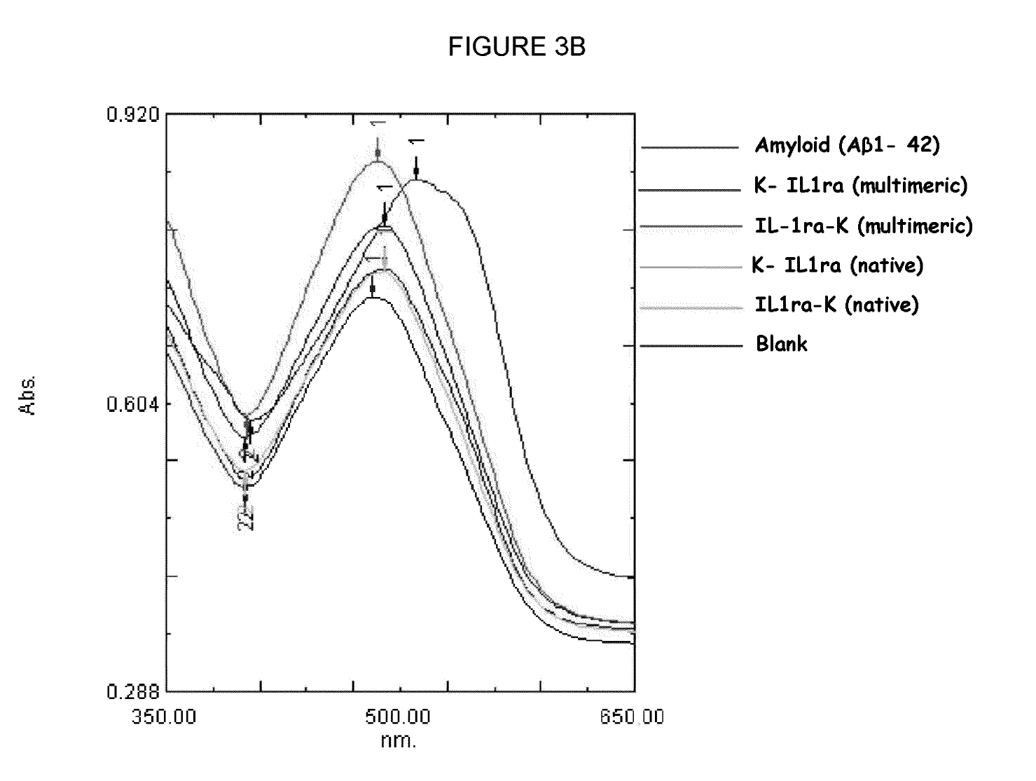

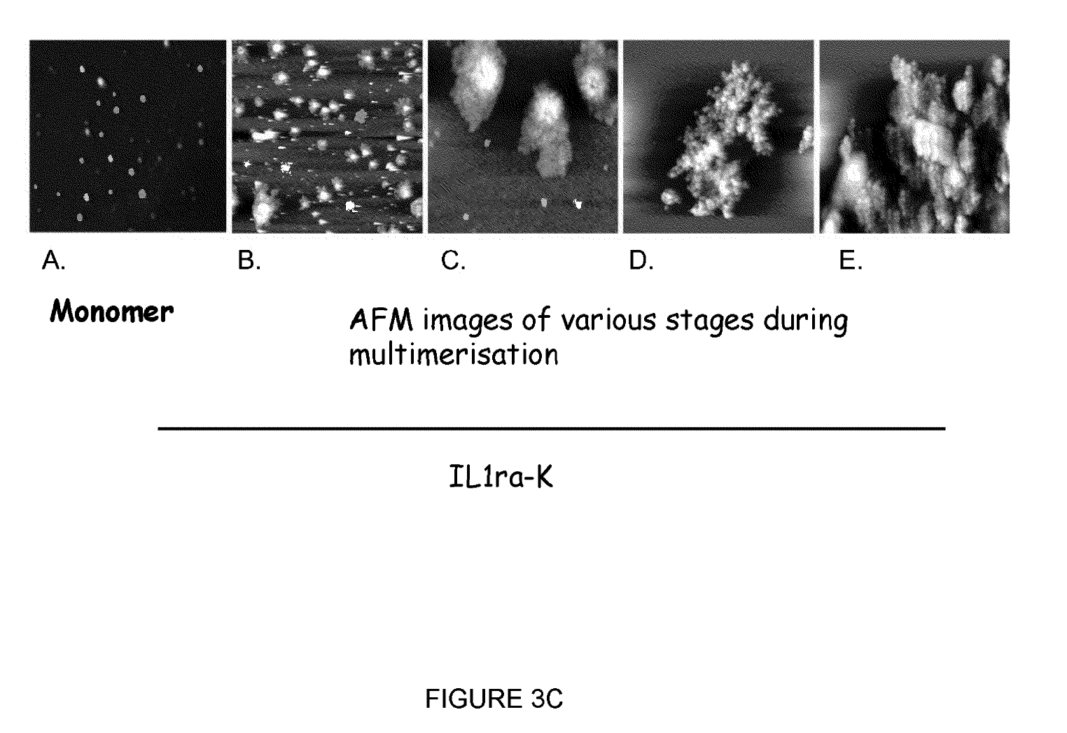

Characterization of multimers using Thioflavin T fluorescence assay is described in Example 3. The observed increase in Thioflavin T fluorescence on binding to IL-1 raK, KIL-1ra and KIL-1raK aggregates (FIG. 3A) was only 3-3.5 fold in comparison to a greater than 100 fold increase in case of Aβ 1-42 which was used as a positive control (FIG. 3A)

Characterization of Multimers using Congo-Red (CR) dye binding (Klunk, W. E., Jacob, R. F. & Mason, R. P. Quantifying amyloid by Congo red spectral shift assay. Methods Enzymol 309, 285-305 (1999)). Like Thioflavin T, CR also binds specifically to the β-sheet rich structures of amyloid and has been used routinely for their detection. CR binding to samples incubated with 50 μM CR in PBS for 1 h at 37° C. was monitored by the red shift in its absorption maximum by scanning 400-600 nm regions. FIG. 3B provides that the multimers exhibit weak binding to CR, whereas fully grown fibers of Aβ 1-42 (positive control) showed significant binding.

Morphology of multimers formed by IL-1raK was assessed by atomic force microscopy (Example 3). Samples were drawn at different time points during the multimerisation process and analyzed morphologically using Atomic Force Microscope (AFM). AFM images magnificently reveal self-association of monomers into nuclei (FIG. 3C panel B), representing the rate limiting step of protein aggregation, which then grow into large protein aggregates of various sizes. A definite molecular organisation seems lacking in the multimers which indicates their amorphous nature. However, a closer look at these images uncovers a remarkable structural arrangement of monomers in the multimeric state. In FIG. 3C panel E monomers can be seen arranged in short stick like fashion and these protein sticks then seem to be bundled into large multimers of various sizes. Fully grown fibres were absent indicating simple clustering of monomers by way of multimerising motif, without much perturbations or rearrangements in the protein structure.

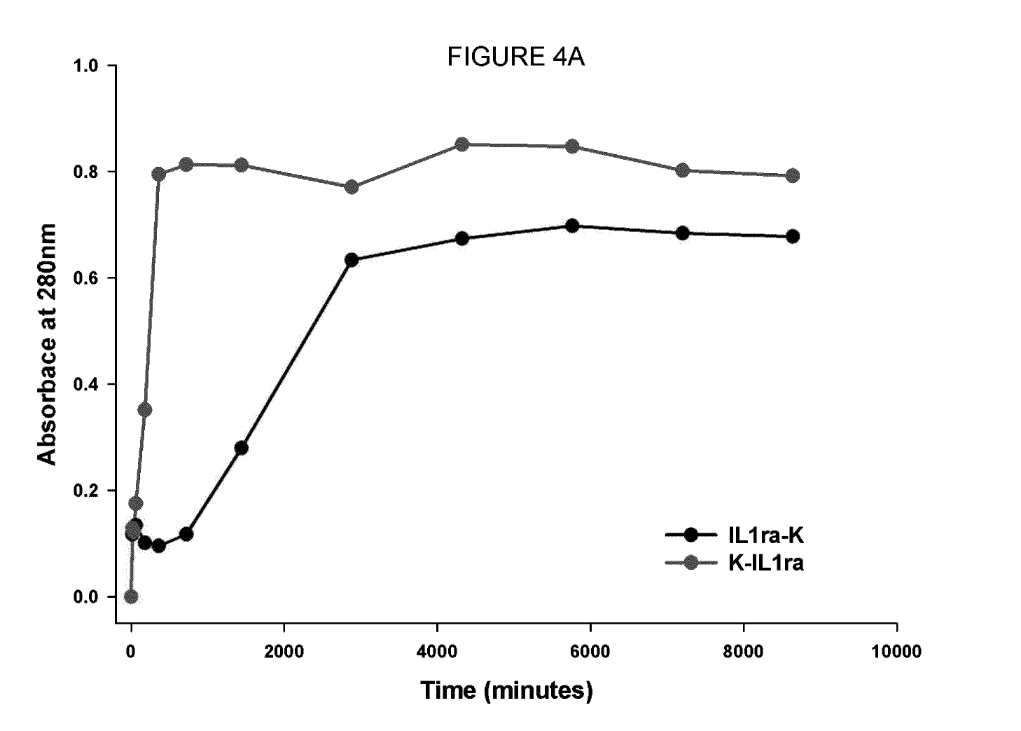

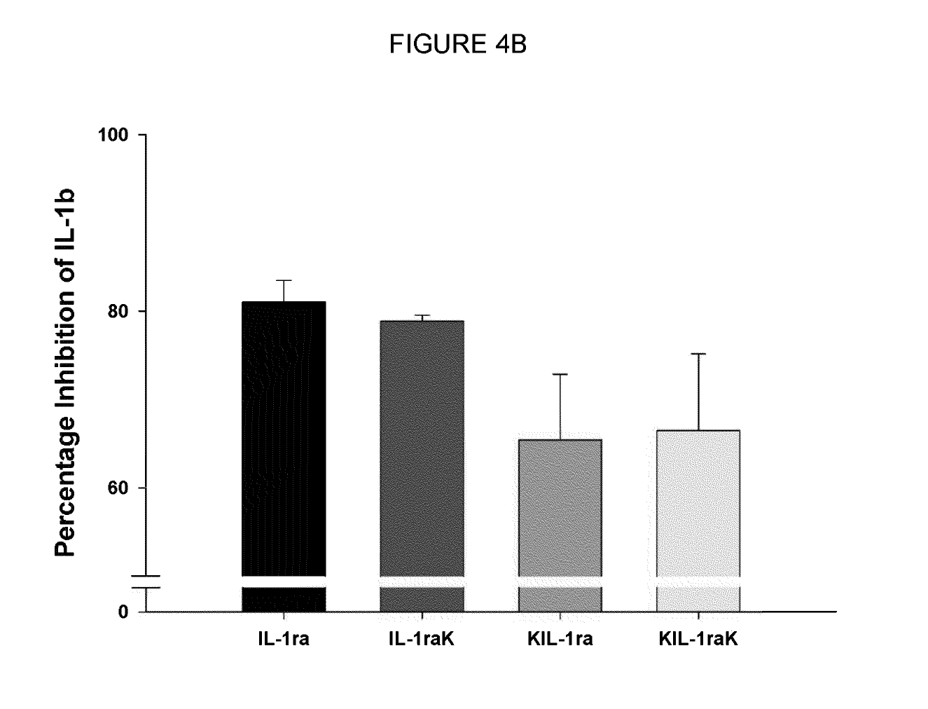

Kinetics of the release of monomers from multimers is shown in Example 4. The multimeric form of IL-1raK, KIL-1ra and KIL-1raK (data not shown) acts as a reservoir for the sustained release of respective monomers (FIG. 4A). The release of IL-1raK, KIL-1ra and KIL-1raK from their respective multimeric forms was noted and is depicted by FIG. 4a. The multimers of the three IL-1ra variants, exhibiting a turbidity of 1.6-2.0 at 405 nm, release monomers at an appreciable rate. A linear increase in the release of respective monomers at 280 nm absorbance over a period of 6±2 days is observed.

Biological activity of monomers released from multimers of IL-1raK, KIL-1ra and KIL-1raK was tested on an IL-1 responsive cell line D10.G4.1 (mouse helper T-cells) which proliferates minimally in response to concanavalin A (con A) in the absence of IL-1. The biological activity of monomeric IL-1raK was comparable to IL-1ra in inhibiting the proliferation of D10 cells as shown in FIG. 4B. KIL-1ra and KIL-1raK had relatively less biological activity and therefore further experiments were performed using the C-terminal tagged isoform of IL-1ra i.e. IL-1raK

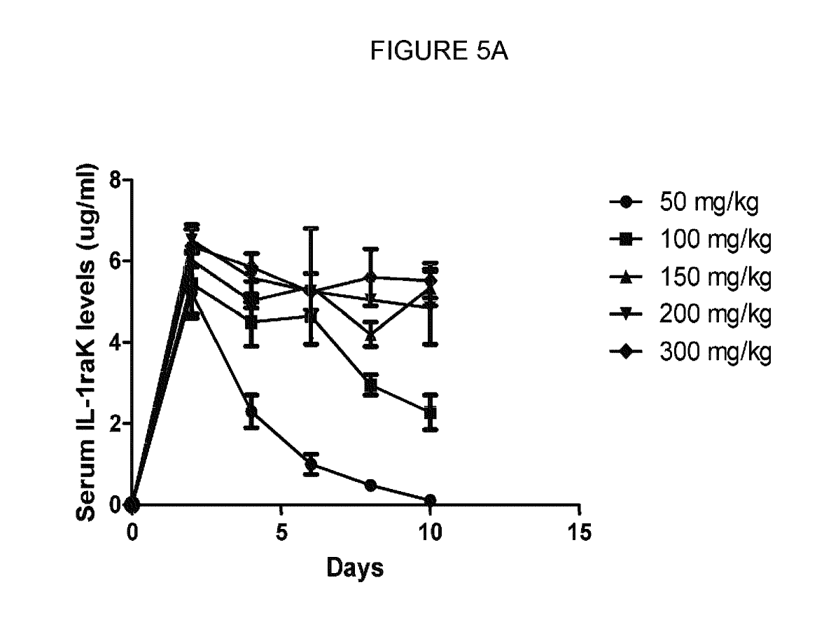

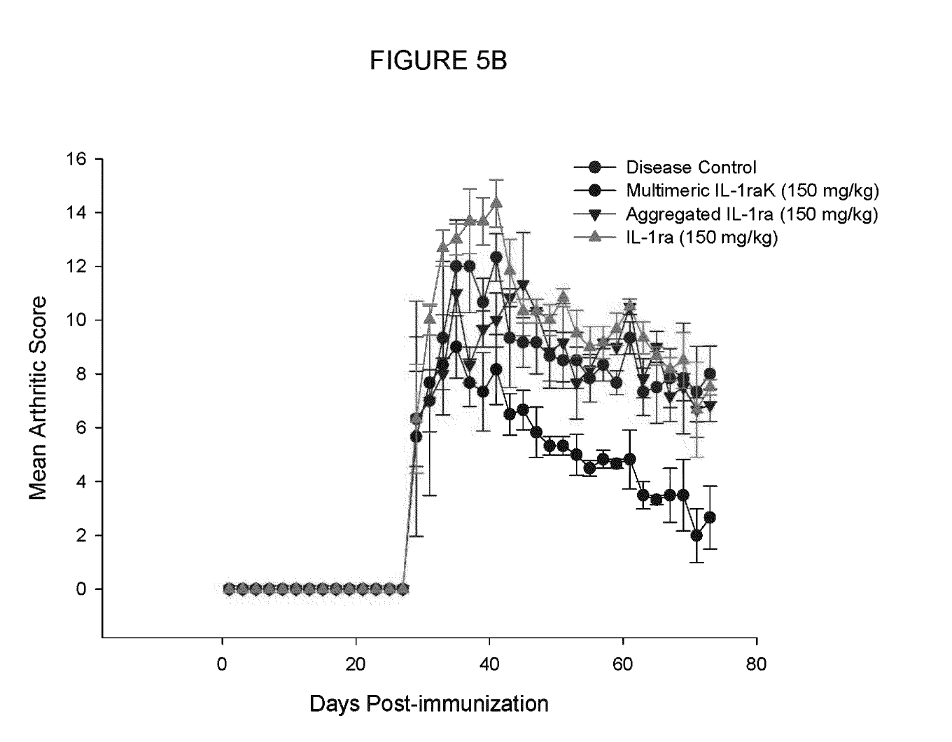

Dose titration of multimeric IL-1raK for treatment of Collagen-induced arthritis in DBA/1J mice is described in Example 5. Dose titration of multimeric IL-1raK was done in healthy DBA/1J mice. Multimeric IL-1raK at doses 50 mg/kg, 100 mg/kg, 150 mg/kg, 200 mg/kg and 300 mg/kg of body weight was injected sub-cutaneously into DBA/1J mice. Blood samples were drawn at regular intervals and analysed for the presence of released IL-1raK by ELISA. A dose dependent sustained release was observed (FIG. 5A). Release duration up to 7 days achieved with dosages 100-200 mg/kg body weight was considered ideal for further experiments. Serum levels were found to be in the range of 4-6 ug/ml. Since IL-1raK is an immunomodulatory molecule therefore, its presence at high levels for prolonged periods is undesirable. Therefore, a release period of maximum 7 days was chosen. Dosages 100-200 mg/kg body weight, were used for further experimentation.

Example 5 also provides the details about treatment of collagen-induced arthritic mice with multimeric IL-1raK. Multimers of IL-1raK were tested in an animal model of experimental autoimmune arthritis in DBA/1J to determine its therapeutic efficacy. Animals with established CIA (on appearance of definitive clinical symptoms i.e. clinical score≧4) were treated with multimeric IL-1raK, monomeric IL-1ra and aggregated IL-1ra at a dose of 150 mg/kg body weight subcutaneously. Clinical symptoms of the disease were scored subjectively on a scale of 0-4. An approximate reduction of 50% in the clinical signs and symptoms of the disease was observed which persisted for ˜5 weeks over an experimental period of ˜11 weeks and ˜35% reduction in clinical score was observed when compared to untreated control. Though, the presence of IL-1raK at the mentioned dose is observed only for around 6-7 days but its pharmacological effects were observed for even longer periods. Protection of joints from early damage by the IL-1raK monomers released from the multimers translates into a significant reduction in disease severity for extended periods. As shown in FIG. 5B clinical score in the IL-1raK treated animals did not increase further after the therapeutic intervention. In contrast, vehicle treated or IL-1ra treated (single sub-cutaneous injection of equivalent dose) or aggregated IL-1 ra treated mice did not show any measurable therapeutic benefit. The results thus indicate that though native IL-1ra can sometimes form aggregates but those aggregates either do not release monomers at all or the monomers released from them are not biologically active. Disease severity was found to reduce even in the disease control, IL-1ra treated and aggregated IL-1ra treated groups after 40 days of intervention which may be attributed to the self limiting pattern of CIA. However, the decrease in case of IL-1raK treated group was significant and more pronounced testifying the in vivo efficacy of constantly released IL-1raK monomers.

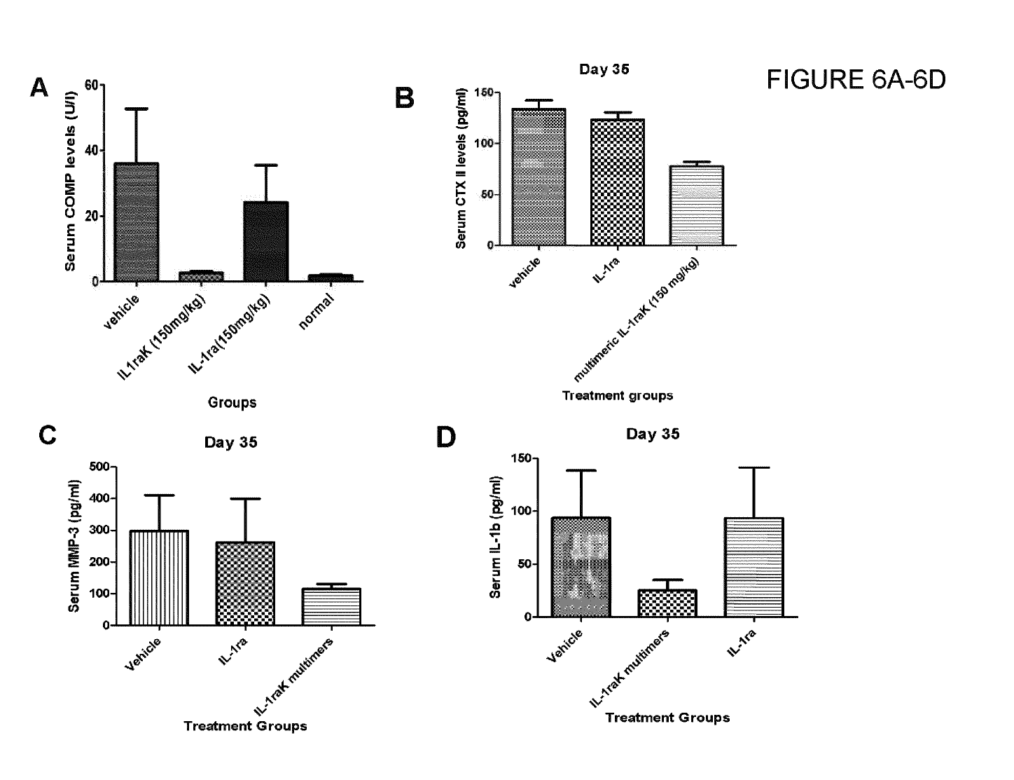

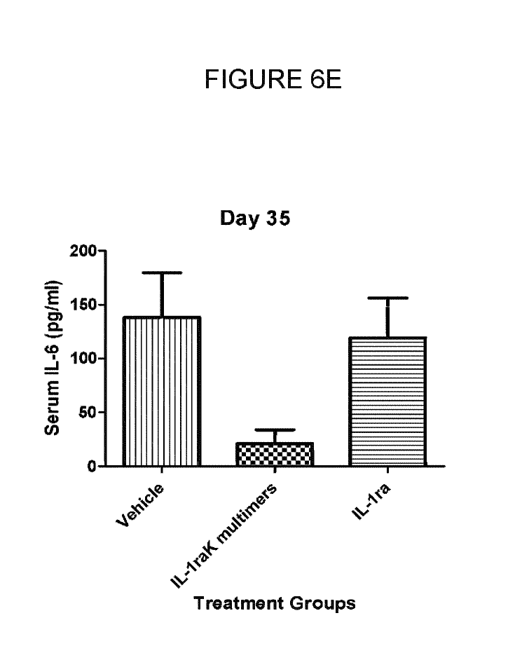

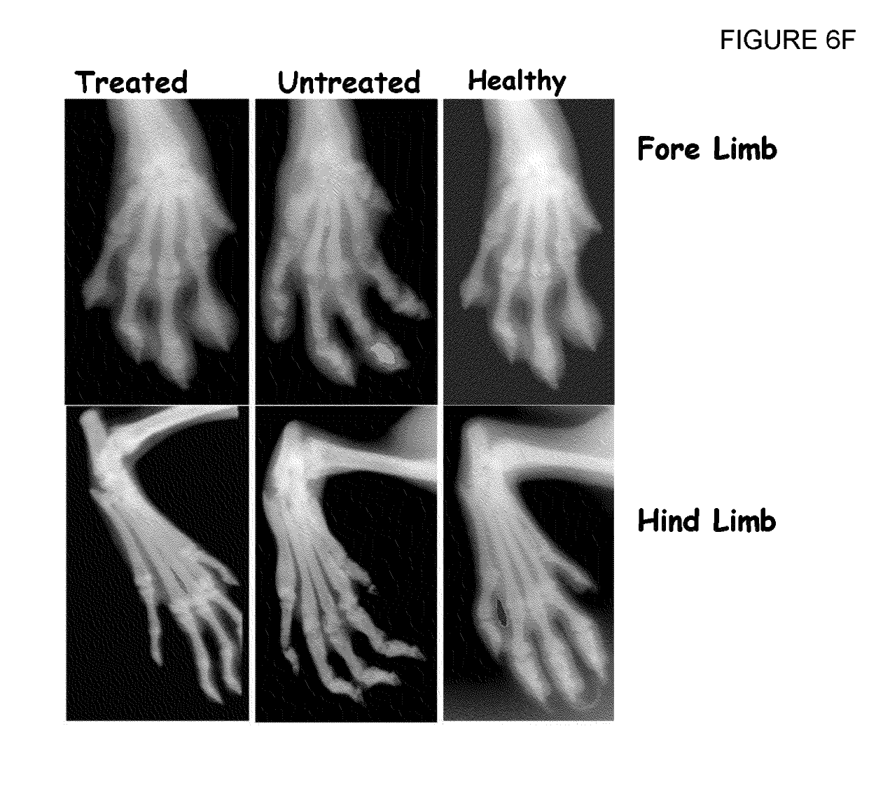

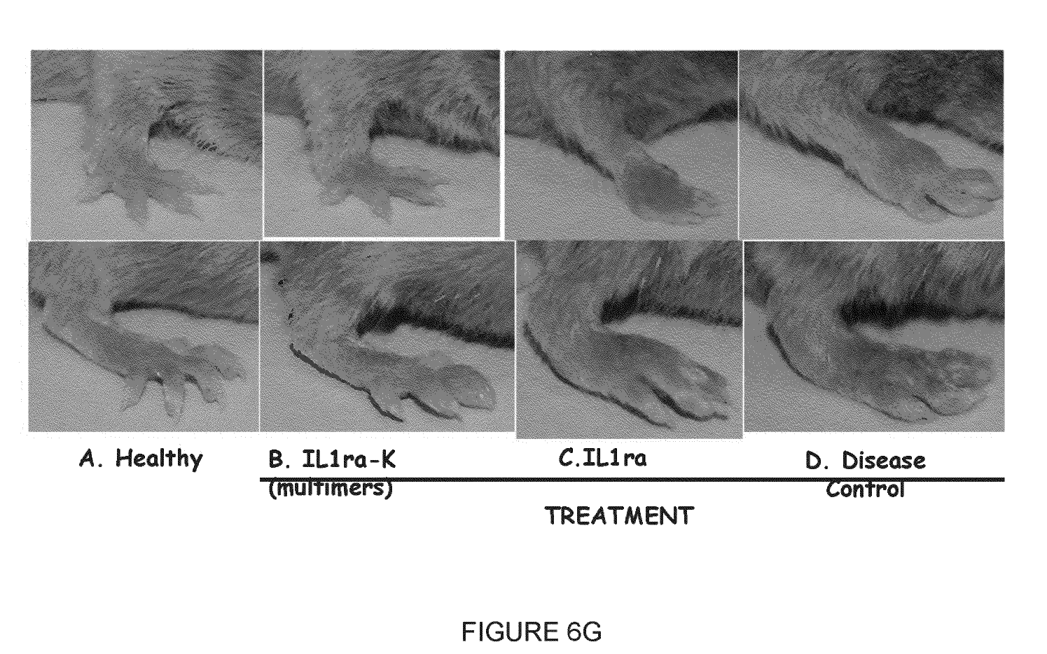

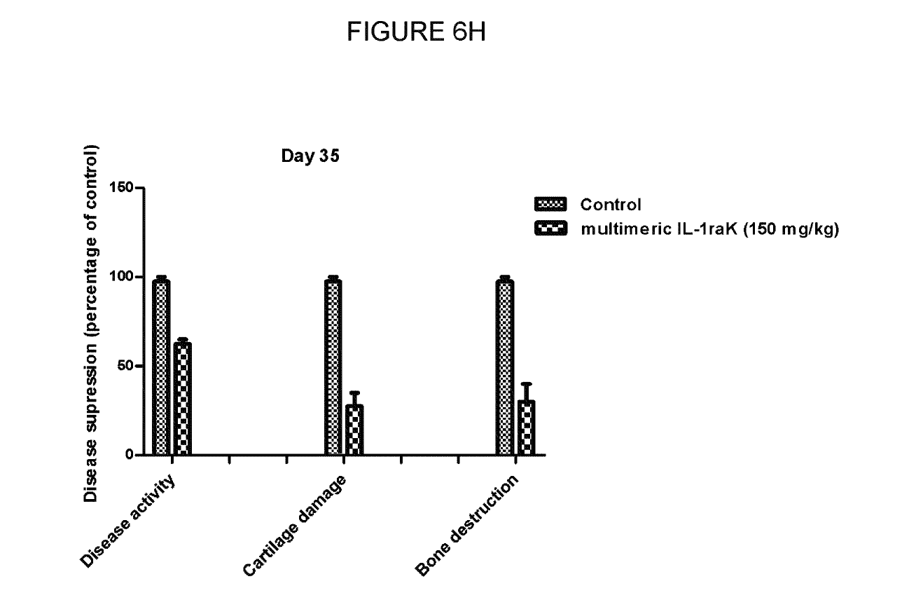

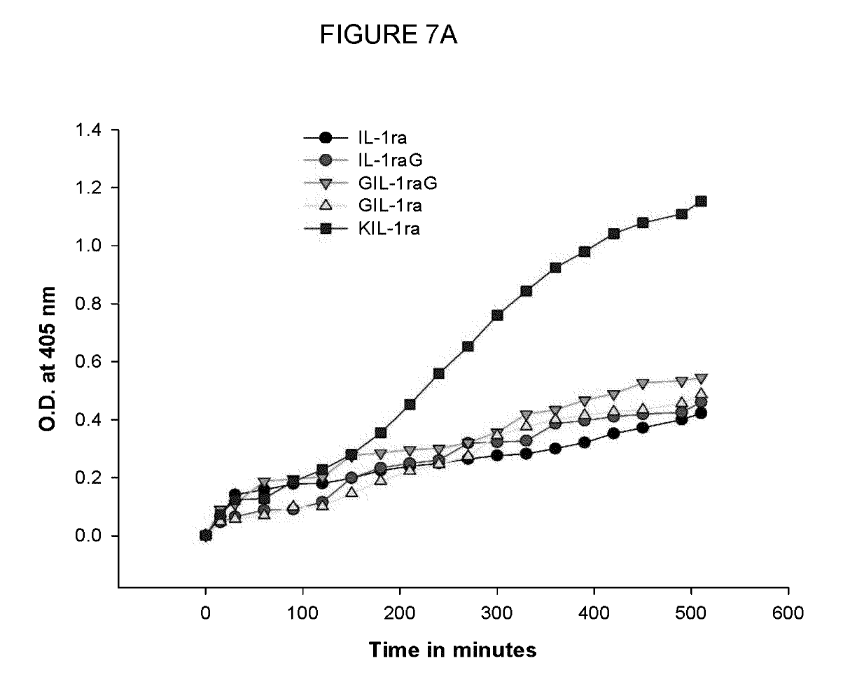

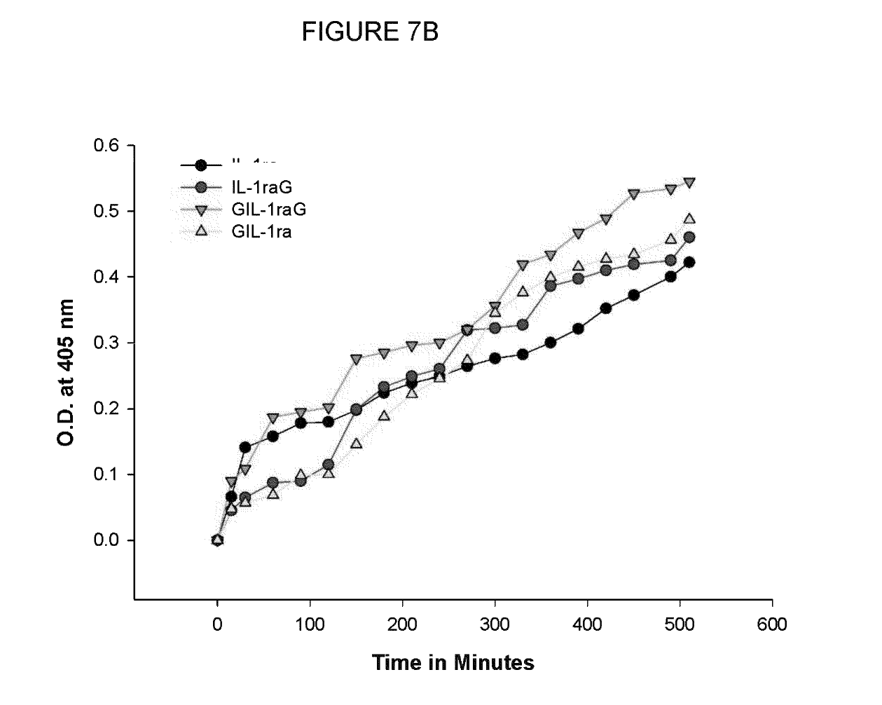

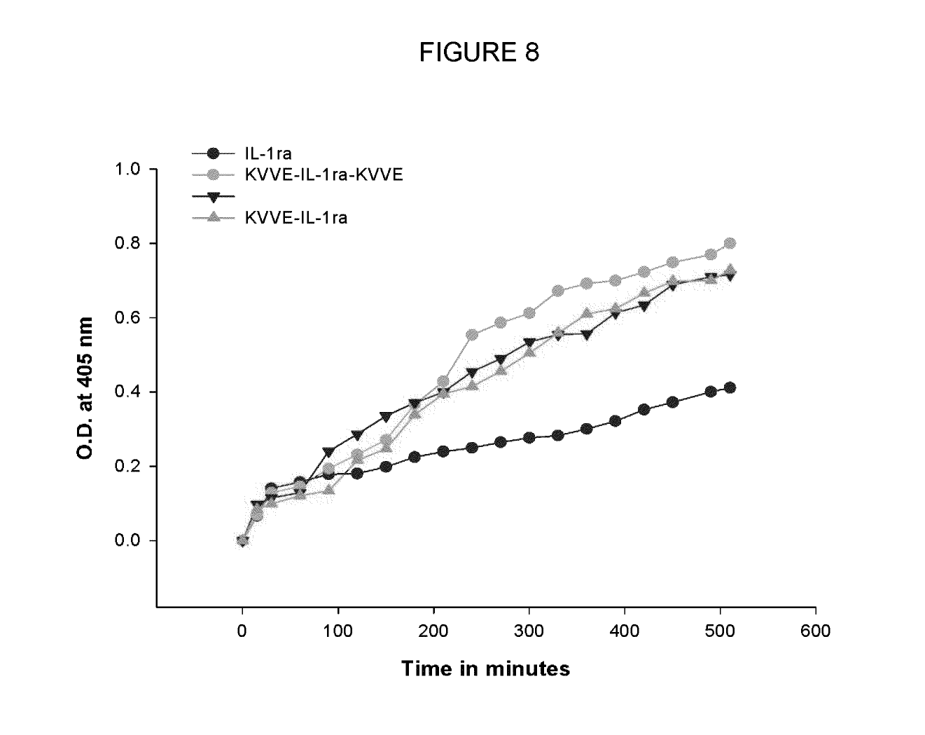

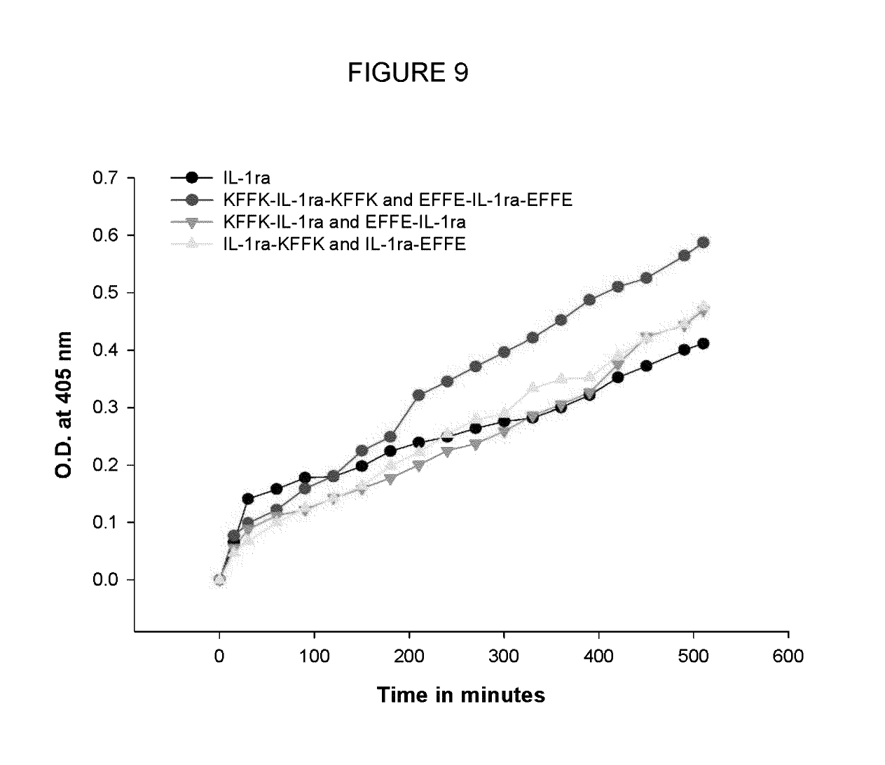

There is a strong correlation between severe cartilage damage and increased serum COMP levels during murine CIA. Serum COMP levels were determined in various groups to identify protection against severe cartilage damage by monomers released from multimeric IL-1raK. An approximate 10 fold decrease in the COMP levels of multimeric IL-1raK treated group can be seen which is still about 1.5-2 fold higher than the levels in non-arthritic control animals (FIG. 6A). A hallmark of rheumatoid arthritis is disruption of the structural integrity of cartilage. Type II collagen is the major organic constituent of cartilage and fragments of type II collagen (CTX-II) are released into circulation during the destructive process. Therefore, serum CTX-II levels were determined in various experimental groups as shown in FIG. 6B. The mean CTX-II levels in animals treated with multimeric IL-1raK were significantly reduced (85±9.2 pg/ml; p<0.05) in comparison to disease controls (FIG. 6B). Animals treated with a single dose of IL-1ra displayed CTX-II levels comparable to untreated animals (FIG. 6B). FIG. 6C shows the levels of matrix degrading enzyme in various experimental groups. The levels of MMP-3 was reduced by 75-80% in the multimeric IL-1raK treatment group (mean levels<120 ng/ml) at day 35 of treatment, while treatment with a single dose of IL-1ra had no effect on the levels of MMP-3.

Effect of multimeric IL-1raK on serum levels of pro-inflammatory cytokines is described in Example 6. Quantification of major pro-inflammatory cytokines indicated that serum levels of IL-1b and IL-6 were significantly reduced in the multimeric IL-1raK treatment group in comparison to disease controls and IL-1ra treated animals as shown in FIGS. 6d and e. Since all these cytokines have a synergistic effect on disease progression and the tissue damage that ensues, therefore, a reduction in their levels is of great importance.

Effect of multimeric IL-1raK treatment on radiographic progression of CIA is shown in FIG. 5G. Radiographic analysis was performed to evaluate joint and bone destruction, a common feature of murine collagen arthritis. Radiographs of the paws were taken at points when maximum effect of the treatment was observed. FIG. 6F shows that even a single dose of multimeric IL-1raK prevents bone destruction by up to 80% as determined by the number of eroded joints. Tarsometatarsal, carpometacarpals (FIG. 6F), metatarsophalangeal and metacarpophalangeal (FIG. 6F) joints of disease controls show severe erosion compared to multimeric IL-1raK treated animals which have distinct joint spacing and outline. Bone deformity to a certain extent can also be seen in the multimeric IL-1raK treatment group but it is significantly less in comparison to disease control animals.

Photographs of fore and hind limbs of one representative animal from each experimental group was taken. As can be clearly seen in FIG. 6G there is marked reduction in inflammation and redness of both paws in the multimeric Il-1raK treatment group while the paws of disease control animal are severely inflamed giving a macroscopic evidence of the effect of treatment on disease severity and progression. The treated animals were also more active and mobile than their untreated or IL-1ra treated counter-parts.

FIG. 6H shows the overall effect of multimeric IL-1raK treatment on various parameters of experimental arthritis such as disease activity, cartilage damage and bone destruction. The treatment group displays a 30-40% improvement in the clinical signs and symptoms of the disease. Cartilage damage and bone destruction, hallmarks of the arthritis are significantly reduced up to 70% in comparison to disease controls as assayed by serum levels of various biomarkers of cartilage damage and radiography.

Example 7 provides details of experiments demonstrating effect of multimeric IL-1raK on DSS induced experimental colitis. Multimeric IL-1raK and IL-1ra at a dose of 150 mg/kg body weight were co-administered with 5% DSS solution. The results summarized in Table 2 demonstrate that multimeric IL-1raK is effective in reducing disease severity and delaying disease progression. The disease activity index (DAI) of animals treated with multimeric IL-1raK is significantly less than disease controls and animals treated with a single dose of IL-1ra.

Example 8 describes induction and treatment of acetaminophen induced liver injury which is an acute model of inflammation. Administration of multimeric IL-1raK at a dose of 150 mg/kg effectively brought down the serum levels of liver enzymes alanine aminotransferase (ALT) and aspartate aminotransferase (AST) and thus further injury to the liver was arrested (Table 3).

Examples 9-13 describe cloning, expression, purification and assessment of additional IL-1ra variants attached to one or more additional distinct and exemplary multimerisation motifs. The motifs GNNQQNY, KVVE, KFFK and EFFE were attached to IL-1ra, including covalently at the N-, C-, or both the N- and C-terminus of native IL-1ra, and multimerisation monitored using turbidimetric assay as previously and herein described.

Unless otherwise defined, all technical and scientific terms used herein have the same meaning as commonly understood by one of ordinary skill in the art to which this invention belongs. Although materials and methods similar to those described herein can be used in practice or testing of the invention, suitable methods and materials are described below. All publications, patent applications, patents, and other references mentioned herein are incorporated by reference in their entirety. In case of conflict, present specification, including definitions, will control. In addition, the materials, methods, and examples are illustrative only and not intended to be limiting.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention. Other features and advantages of the invention will be apparent from the following detailed description, examples and claims.

### Example 1

### Cloning, Expression and Purification of Human IL-1 Receptor Antagonist and its Variants

Poly(A) ⁺RNA isolated from THP-1 monocytic cells (ATCC, USA) stimulated with 1 mg/ml LPS and 100 ng/ml PMA was reverse transcribed using oligo (dT)₁₈ primers and random hexamers. The cDNA thus obtained was amplified by polymerase chain reaction (PCR) using 5′- and 3′-primers corresponding to the coding sequence of IL-1ra (accession no. NM_{—}173842). The primer sequences are as follows:

KIL-1raK Forward Primer
5′-AAGCTTTGAAATTTTTTGAACGACCCTCTGGGAGAAAATCC-3′ (SEQ ID NO: 32)
KIL-1raK Reverse Primer
5′-AATTCTTATTTAAAAAATTCCTCGTCCTCCTGGAAGTAGAATTTGG-3′ (SEQ ID NO: 33)

The primers for IL-1ra do not include the underlined nucleotide bases present both in the forward and reverse primers. The primers for IL-1raK do not include the underlined nucleotide bases present in the forward primer. The primers for KIL-1ra do not include the underlined nucleotide bases present in the reverse primer.

Additional sequences corresponding to the multimerising motif KFFE and the affinity tag for protein purification were incorporated into the primers. The amplified product was cloned into pPAL7 expression vector (carrying an ampicillin resistance gene) using restriction endonuclease free cloning strategy. The IL-1ra fusion proteins with an 8kD N-terminal Profinity eXact tag (recognized by a mutant subtilisin protease S189) were expressed using the Bio-Rad Profinity eXact protein purification system. The correct sequence of the cloned IL-1ra (accession no. NM_{—}173842) and its variants (IL-1raK, KIL-1ra, KIL-1raK) was verified by DNA sequencing.

The nucleotide sequence of IL-1ra is as follows:

5′CGACCCTCTGGGAGAAAATCCAGCAAGATGCAAGCCTTCAGAATCTGGGATGTTAACCAGAAGA
CCTTCTATCTGAGGAACAACCAACTAGTTGCTGGATATTGCAAGGACCAAATGTCAATTTAGAAGA
AAAGATAGATGTGGTACCCATTGGCCTCATGCTCTGTTCTTGGGAATCCATGGAGGGAAGATGTGC
CTGTCCTGTTCAAGTCTGGTGATGAGACCAGACTCCAGCTGGAGGCAGTTAACATCACTGACCTGA
GCGAGAACAGAAAGCAGGACAAGCGCTTCGCCTTCATCCGCTCAGACAGCGGCCCCACCACCAGT
TTTGAGTCTGCCGCCTGCCCCGGTTGGTTCCTCTGCACAGCGATGGAAGCTGACCAGCCCGTCAGC
CTCACCAATATGCCTGACGAAGGCGTCATGGTCACCAAATTCTACTTCCAGGAGGACGAGTAGTA3′
(SEQ ID NO: 34)

IL-1raK, KIL-1ra and KIL-1raK contain AAATTTTTTGAA (SEQ ID NO:35) corresponding to the multimerising motif KFFE at the C-terminus, N-terminus and at both termini respectively.

The cloned cDNA was placed under the control of a T7lac promoter. Unmodified plasmid carrying the ampicillin resistance gene served as control. Plasmids pPAL7 IL-1ra, pPAL7 IL-1raKFFE, pPAL7 KFFEIL-1ra and pPAL7 KFFEIL-1raKFFE were amplified in E. coli DH5α and purified using commercially available plasmid purification kit (Sigma). For expression, the plasmids containing IL-1ra, IL-1raK, KIL-1 ra, KIL-1raK genes were cloned into E. coli BL21 (DE3) cells. Protein expression was induced using isopropyl-β-D-thiogalactopyranoside (IPTG) and the expressed protein was purified by affinity chromatography using Bio-scale mini Profinity eXact FPLC columns from Bio-Rad. Identity of the expressed protein was established by western blot using anti-IL-1ra antibody.

### Example 2

### Multimerisation of IL-1raK, KIL-1ra and KIL-1raK

Multimerisation of IL-1raK, KIL-1ra and KIL-1raK was performed under isothermal conditions. 1 ml of 20 mg/ml IL-1raK, KIL-1ra, KIL-1raK in 50 mM sodium phosphate buffer pH 6.0 was aliquoted into a 2 ml microcentrifuge tube and kept at 37° C. with shaking at 200 rpm. Kinetics of multimerisation was followed by measuring optical density (OD) at 405 nm at every 30 min interval caused by increase in turbidity. The multimers were also characterized by Thioflavin T and Congo red dye binding assay.

### Example 3

### Characterization of Multimeric Form of IL-1raK, KIL-1ra and KIL-1raK

Thioflavin T Fluorescence Assay

Thioflavin T binding assays were performed in a Jobin Yvon Fluoromax spectrofluorimeter using an excitation and emission slit width of 5 nm. Samples were excited at 420 nm and emission was recorded in the range of 450-600 nm. Prior, to each fluorescence measurement, samples were incubated with 50 μM Thioflavin T for 15 minutes at 25° C. in dark. Data were corrected for blank and inner filter effect using the following equation:

Fc=F antilog[(Aₑₓ+Aₑₘ)/2]

where, Fc is the corrected fluorescence, F is measured fluorescence, and Aₑₓ and Aₑₘ, are the absorbance of the solutions at the excitation and emission wavelengths, respectively.

Congo Red (CR) Binding Assay

Samples were incubated with 190 μl of Congo red dye (50 μM) at 37° C. for 1 hour in dark. The CR binding was observed by monitoring absorption spectra of the sample at 400-700 nm using Shimadzu UV 2450 spectrophotometer. The amount of CR bound to multimers was estimated as reported earlier (2). The amount of bound CR was calculated using the following equation:

Moles of CR bound/L of amyloid suspension=A_{540 nm}/25,295−A_{477 nm}/46,306.

Atomic Force Microscopy

Pico plus Atomic Force Microscope (Agilent Technologies) was used in non-contact mode for imaging. Samples were withdrawn from the multimersation reaction mixture at various time points, centrifuged at 10,000 rpm for 10 minutes at 4° C. The resulting pellet was resuspended in 100 μl water and immobilized on freshly cleaved mica for 2 minutes. Samples were washed with ultrapure water, dried and subjected to AFM analysis.

### Example 4

### In-Vitro Release Assay for Multimeric Forms of IL-1raK, KIL-1ra and KIL-1raK

Multimers of different time points were centrifuged at 10 k rpm for 10 minutes, washed with 1× cold PBS, resuspended in 5 ml 1×PBS and kept at 37° C., shaking 200 rpm. Aliquots were withdrawn at regular intervals, centrifuged, and absorbance of the supernatant was measured at 280 nm.

In Vitro Assay for Bioactivity of Monomers Released from the Multimeric Forms of IL-1raK, KIL-1ra and KIL-1raK

IL-1 responsive mouse T helper D10.G4.1 cell line was purchased from ATCC. Assay to check the bioactivity of protein released from multimeric variants of IL-1ra was performed as described by McIntyre et al (1991). Briefly, 2×10⁴ cells suspended in RPMI 1640 containing 10% FCS, 5×10⁻⁵ M β-ME and 2.5 μg/ml Con A were seeded in 96-well flat bottom tissue culture plates. Released IL-1ra was added to triplicate cultures 1 hour before the addition of IL-1β. The plates were incubated for 3 days at 37° C. in a humidified atmosphere of 5% CO₂. After 3 days, cells were pulsed with 0.5 μCi of [³H] thymidine and incubated for an additional 18 hours. The cells were harvested onto glass fiber filters and level of thymidine incorporation determined using liquid scintillation counter.

### Example 5

### Treatment of Arthritis

Animals

8 week old male DBA/1J mice were used in the study. Animals were allowed to acclimate for 2 weeks prior to the experiments: All animals were given ad libitum access to food and water. The experimental protocol and animal handling was strictly in accordance with the Institutional Animal Ethics committee of National Institute of Immunology, New Delhi, India.

Dose Titration of Multimeric IL-1raK

8-10 week old healthy DBA/1J mice were injected sub-cutaneously with various dosages of multimeric IL-1raK namely, 50 mg/kg, 100 mg/kg, 150 mg/kg, 200 mg/kg and 300 mg/kg body weight (n=10 per group). Blood samples were withdrawn every alternate day from 2 animals from each group. Serum was separated and amount of IL-1raK released was quantified by using human IL-1ra ELISA kit from RnD systems.

Induction and Assessment of Arthritis

Autoimmune experimental arthritis was induced in male DBA/1J mice using bovine type II collagen. Bovine type II collagen was dissolved in 10 mM acetic acid and emulsified in Complete Freund's Adjuvant (CFA; 4 mg/ml) to a final concentration of 5 mg/ml. Animals were immunized with 50 μl of the emulsion intradermally at the base of the tail. Disease usually developed 18-25 days post immunization and the day swelling or erythema in the paws was observed, it was recorded as day 1.

Autoimmune experimental arthritis as mentioned above may further comprise of Oil-induced arthritis, proteoglycan induced arthritis (PGIA) and may also further comprise of transgenic mice models such as K/BxN mice, SKG mice (ZAP 70 mutation), TNFα and IL-1ra−/− transgenic mice.

Diseased animals were assessed every alternate day to monitor disease progression. Assessment of the disease was based on a subjective scoring system. Each paw was evaluated and scored individually on a scale of 0-4. The scoring system used was as follows: 0=No evidence of erythema and swelling, 1=Mild erythema or swelling (detectable arthritis), 2=Moderate erythema and swelling, 3=Significant erythema and swelling encompassing entire paw, 4=Maximal swelling with or without limb distortion.

Treatment

Animals with established disease i.e. clinical scores≧4, were divided into 3 experimental groups with 3 animals per group. Group I comprised of vehicle treated animals which served as disease controls. Group II and III consisted of animals receiving single sub-cutaneous injection of IL-1ra and multimeric IL-1raK (150 mg/kg body weight) respectively.

### Example 6

### Serum Levels of Biomarkers of Tissue Damage

Mice serum samples were collected at day 35 and analysed for various biomarkers of tissue damage such as Cartilage Oligmeric Matrix Protein (COMP), CTX II and MMP-3. COMP ELISA was purchased from AnaMar AB, Sweden. CTX II levels determined using serum preclinical cartilaps ELISA from Immunodiagnostics systems. Serum levels of MMP-3 quantified by ELISA from RnD systems.

Serum Levels of Pro-Inflammatory Cytokines

Serum samples were collected at day 35 of treatment and levels of pro-inflammatory cytokines were determined using multiplex cytokine kits from Millipore.

X-Ray Analysis

X-ray radiographs were taken (Kodak In vivo Imaging System FX Pro) of one fore and hind limb. The severity of bone erosion was ranked as described by Seeuws et al (2010) using a modified version of Larsen scoring method: 0=normal; 1=slight abnormality with any one or two of the exterior metatarsal bones showing slight bone erosion; 2=definite early abnormality with any of the metatarsal or tarsal bones showing bone erosion; 3=medium destructive abnormality with any of the metatarsal or any one of the tarsal bones showing definite bone erosion; 4=severe destructive abnormality with all the metatarsal bones showing definite bone erosion and at least one of the tarsometatarsal joints being completely eroded, leaving some bony joint outlines partly preserved; 5=mutilating abnormality with no bony outlines that can be deciphered.

### Example 7

### Treatment of Colitis

Animals

8-10 weeks old Balb/cJ mice were used for experiments. Animals were allowed to acclimate for 2 weeks prior to the experiments. All animals were given ad libitum access to food and water. The experimental protocol and animal handling was strictly in accordance with the Institutional Animal Ethics committee of National Institute of Immunology, New Delhi, India.

Induction and Assessment of Experimental Colitis

Mice were weighed and divided into three experimental groups namely, disease control, multimeric IL-1raK treated and IL-1ra treated, such that the average weight per experimental group was same. Each experimental group received 5% dextran sodium sulphate (DSS) in tap water for 7 days along with a single subcutaneous injection of various treatments such as PBS (vehicle), IL-1ra and multimeric IL-1raK, at the start of experiments. The general condition of mice in each experimental group was scored by scoring the extent of body weight loss, stool guaiac positivity or gross bleeding and stool consistency. The scoring system known as disease activity index (DAI) is as described in table 1 (Hamamoto N et al. Clin Exp Immunol. 1999 September; 117(3): 462-468))

### Example 8

### Treatment of Induced Liver Injury (AILI)

Animals

8-10 weeks old male C57BL/6J mice were used in the study. Animals were allowed to acclimate for 2 weeks prior to the experiments. All animals were given ad libitum access to food and water. The experimental protocol and animal handling was strictly in accordance with the Institutional Animal Ethics committee of National Institute of Immunology, New Delhi, India.

Induction and Assessment of Acetaminophen (APAP) Induced Liver Injury (AILI)

AILI was induced in mice by a single intraperitoneal injection of acetaminophen (300 mg/kg). Whole blood samples were collected at regular intervals to determine serum activities of liver enzymes ALT and AST.

Treatment

Diseased animals were divided into 3 groups and treated with PBS (vehicle), IL-1ra (150 mg/kg), or multimeric IL-1raK (150 mg/kg).

TABLE 1
Scoring system for monitoring disease
progression in experimental colitis:

| Score | % weight | Stool | occult/ |
|---|---|---|---|
| Bleeding | loss | consistency | gross |
| 0 | None | Normal | Normal |
| 1 | 1-5 | loose stools | guaiac |
| 2 | 5-10 | positive | |
| 3 | 10-15 | Diarrhoea | gross |
| 4 | >15 | bleeding | |

The disease activity index is calculated as follows:

DAI=(score of weight loss+stool consistency+bleeding)/3.

TABLE 2
Disease Activity Index (DAI) of various experimental
groups in a mice model of experimental colitis.

| Group | Disease Activity Index (DAI) |
|---|---|
| Disease Control (untreated) | 2.586 ± 0.197 |
| Multimeric IL-1raK treated | 1.692 ± 0.202 |
| IL-1ra treated | 2.457 ± 0.145 |

TABLE 3
Serum levels of liver enzymes alanine aminotransferase (ALT)
aspartate aminotransferase (AST) in a mice model of AILI.

| Group | ALT (IU/L) | AST (IU/L) |
|---|---|---|
| Disease control | 3347 ± 128 | 4926 ± 241 |
| IL-1ra | 3169 ± 175 | 3456 ± 197 |
| Multimeric IL-1raK | 1127 ± 88 | 498 ± 25 |

SEQ ID NO: 1: Amino acid sequence of IL-1raK (156 a.a.; 17.68 kDa)
RPSGRKSSKMQAFRIWDVNQKTFYLRNNQLVAGYLQGPNVNLEEKIDVVPIEPHALFLGIHGGKMCLS
CVKSGDETRLQLEAVNITDLSENRKQDKRFAFIRSDSGPTTSFESAACPGWFLCTAMEADQPVSLTNMP
DEGVMVTKFYFQEDEEFFK
SEQ ID NO: 2: Amino acid sequence of KIL-1ra (156 a.a.; 17.68 kDa)
KFFERPSGRKSSKMQAFRIWDVNQKTFYLRNNQLVAGYLQGPNVNLEEKIDVVPIEPHALFLGIHGGK
MCLSCVKSGDETRLQLEAVNITDLSENRKQDKRFAF1RSDSGPTTSFESAACPGWFLCTAMEADQPVSL
TNMPDEGVMVTKFYFQEDE
SEQ ID NO: 3: Amino acid sequence of KIL-1raK (160 a.a.; 18.23 kDa)
KFFERPSGRKSSKMQAFRIWDVNQKTFYLRNNQLVAGYLQGPNVNLEEKIDVVPIEPHALFLGIHGGK
MCLSCVKSGDETRLQLEAVNITDLSENRKQDKRFAF1RSDSGPTTSFESAACPGWFLCTAMEADQPVSL
TNMPDEGVMVTKFYFQEDEEFFK
SEQ ID NO. 4: Amino acid sequence of IL-1ra (152 a.a. ; 17.13 kDa)
RPSGRKSSKMQAFRIWDVNQKTFYLRNNQLVAGYLQGPNVNLEEKIDVVPIEPHALFLGIHGGKMCLS
CVKSGDETRLQLEAVNITDLSENRKQDKRFAFIRSDSGPTTSFESAACPGWFLCTAMEADQPVSLTNMP
DEGVMVTKFYFQEDE

### Example 9

### Cloning, Expression and Purification of Additional IL-1ra Variants

Poly(A) ⁺RNA isolated from THP-1 monocytic cells (ATCC, USA) stimulated with 1 mg/ml LPS and 100 ng/ml PMA was reverse transcribed using oligo (dT)₁₈ primers and random hexamers. The cDNA thus obtained was amplified by polymerase chain reaction (PCR) using 5′- and 3′-primers corresponding to the coding sequence of IL-1ra (accession no. NM_{—}173842). The primer sequences are as follows:

GIL-1raG Forward Primer
5-′AAGCTTTGGGCAACAACCAACAAAACTATCGACCCTCTGGGAGAAAATCC-3′ (SEQ ID NO: 36)
GIL-1raG Reverse Primer
5′-AATTCTTAATAGTTTTGTTGGTTGTTGCCCTCGTCCTCCTGGAAGTAGAATTTGG-3′ (SEQ ID
NO: 37)

The primers for IL-1raG do not include the underlined nucleotide bases present in the forward primer. The primers for GIL-1ra do not include the underlined nucleotide bases present in the reverse primer.

Additional sequences corresponding to the multimerising motif GNNQQNY and the affinity tag for protein purification were incorporated into the primers. The amplified product was cloned into pPAL7 expression vector (carrying an ampicillin resistance gene) using restriction endonuclease free cloning strategy. The IL-1ra fusion proteins with an 8kD N-terminal Profinity eXact tag (recognized by a mutant subtilisin protease S189) were expressed using the Bio-Rad Profinity eXact protein purification system. The correct sequence of the cloned variants of IL-1ra bearing the motif GNNQQNY (IL-1raG, GIL-1ra, GIL-1raG) was verified by DNA sequencing.

The cloned cDNA was placed under the control of a T7lac promoter. Unmodified plasmid carrying the ampicillin resistance gene served as control. Plasmids pPAL7 IL-1raG, pPAL7 GIL-1ra, and pPAL7 GIL-1raG were amplified in E. coli DH5α and purified using commercially available plasmid purification kit (Sigma). For expression, the plasmids containing GIL-1ra, IL-1 raG and GIL-1raG genes were cloned into E. coli BL21 (DE3) cells. Protein expression was induced using isopropyl-β-D-thiogalactopyranoside (IPTG) and the expressed protein was purified by affinity chromatography using Bio-scale mini Profinity eXact FPLC columns from Bio-Rad. Identity of the expressed protein was established by western blot using anti-IL-1ra antibody.

### Example 10

### Multimerisation of IL-1raG, GIL-1ra and GIL-1raG

Multimerisation of IL-1raG, GIL-1ra and GIL-1raG was performed under isothermal conditions. 1 ml of 20 mg/ml IL-1raG, GIL-1ra, GIL-1raG in 50 mM sodium phosphate buffer pH 6.0 was aliquoted into a 2 ml microcentrifuge tube and kept at 37° C. with shaking at 200 rpm. Kinetics of multimerisation was followed by measuring optical density (OD) at 405 nm at every 30 min interval caused by increase in turbidity.

Multimerisation of IL-1ra, GIL-1ra, IL-1raG and GIL-1raG was monitored using turbidimetric assay. Briefly, 1 ml of 20 mg/ml solution of various IL-1ra variants in 50 mM phosphate buffer pH6.0 was agitated at 37° C. at 200 rpm and samples were drawn at every 30 minute interval. The multimerisation profile of GIL-1ra, IL-1raG and GIL-1raG was more or less similar to IL-1ra indicating failure of the motif GNNQQNY in bringing about protein multimerisation at the mentioned conditions and during the observed incubation period. On the other hand, KIL-1ra which was used as a positive control displayed significant multimerisation under similar conditions.

SEQ ID NO: 5: Amino acid sequence of IL-1raG (159 a.a.; 17.95 kDa)
RPSGRKSSKMQAFRIWDVNQKTFYLRNNQLVAGYLQGPNVNLEEKIDVVPIEPHALFLGIHGGKMCLS
CVKSGDETRLQLEAVNITDLSENRKQDKRFAFIRSDSGPTTSFESAACPGWELCTAMEADQPVSLTNMP
DEGVMVTKFYFQEDEGNNQQNY
SEQ ID NO: 6: Amino acid sequence of GIL-1ra (159 a.a.; 17.95 kDa)
GNNQQNYRPSGRKSSKMQAFRIWDVNQKTFYLRNNQLVAGYLQGPNVNLEEK1DVVPIEPHALFLGIH
GGKMCLSCVKSGDETRLQLEAVNITDLSENRKQDKRFAFIRSDSGPTTSFESAACPGWELCTAMEADQ
PVSLTNMPDEGVMVTKFYFQEDE
SEQ ID NO: 7: Amino acid sequence of GIL-1raG (166 a.a.; 18.76 kDa)
GNNQQNYRPSGRKSSKMQAFRIWDVNQKTFYLRNNQLVAGYLQGPNVNLEEKIDVVPIEPHALFLGIH
GGKMCLSCVKSGDETRLQLEAVNITDLSENRKQDKRFAFIRSDSGPTTSFESAACPGWFLCTAMEADQ
PVSLTNMPDEGVMVTKFYFQEDEGNNQQNY

### Example 11

### Cloning, Expression and Purification of Additional IL-1ra Variants

Fusion proteins of IL-1ra with multimerising motifs KVVE, KFFK and EFFE were cloned, expressed and purified using the same approach as described previously. The primer sequences for the above mentioned variants are as follows:

KVVE-IL-1ra Forward Primer
5-′AAGCTTTGAAAGTGGTGGAACGACCCTCTGGGAGAAAATCC-3′ (SEQ ID NO: 38)
KVVE-IL-1ra Reverse Primer
5′-AATTCTTATTTCACCACTTCCTCGTCCTCCTGGAAGTAGAATTTGG-3′ (SEQ ID NO: 39)
KFFK-IL-1ra Forward Primer
5-′AAGCTTTGAAATTTTTTAAACGACCCTCTGGGAGAAAATCC-3′ (SEQ ID NO: 40)
KFFK-IL-1ra Reverse Primer
5′-AATTCTTATTTAAAAAATTTCTCGTCCTCCTGGAAGTAGAATTTGG-3′ (SEQ ID NO: 41)
EFFE-IL-1ra Forward Primer
5-′AAGCTTTGGAATTTTTTGAACGACCCTCTGGGAGAAAATCC-3′ (SEQ ID NO: 42)
EFFE-IL-1ra Reverse Primer
5′-AATTCTTATTCAAAAAATTCCTCGTCCTCCTGGAAGTAGAATTTGG-3′ (SEQ ID NO: 43)

The primers for IL-1 ra-KVVE do not include the underlined nucleotide bases present in the KVVE-IL-1ra forward primer. The primers for KVVE-IL-1ra do not include the underlined nucleotide bases present in the KVVE-IL-1ra reverse primer. The primers for IL-1ra-KFFK do not include the underlined nucleotide bases present in the KFFK-IL-1ra forward primer. The primers for KFFK-IL-1ra do not include the underlined nucleotide bases present in the KFFK-IL-1ra reverse primer. The primers for IL-1ra-EFFE do not include the underlined nucleotide bases present in the EFFE-IL-1ra forward primer. The primers for EFFE-IL-1ra do not include the underlined nucleotide bases present in the EFFE-IL-1ra reverse primer.

Additional sequences corresponding to the multimerising motifs KVVE, KFFK and EFFE and the affinity tag for protein purification were incorporated into the primers. The amplified product was cloned into pPAL7 expression vector (carrying an ampicillin resistance gene) using restriction endonuclease free cloning strategy. The IL-1ra fusion proteins with an 8kD N-terminal Profinity eXact tag (recognized by a mutant subtilisin protease S 189) were expressed using the Bio-Rad Profinity eXact protein purification system. The correct sequence of the cloned variants of IL-1 ra bearing the motifs KVVE (IL-1ra-KVVE, KVVE-IL-1ra, KVVE-IL-1ra-KVVE), KFFK (KFFK-IL-1ra, IL-1ra-KFFK, KFFK-IL-1ra-KFFK) and EFFE (EFFE-IL-1ra, IL-1ra-EFFE, EFFE-IL-1ra-EFFE) was verified by DNA sequencing.

The cloned cDNA was placed under the control of a T7lac promoter. Unmodified plasmid carrying the ampicillin resistance gene served as control. Plasmids pPAL7 IL-1ra-KVVE, pPAL7 KVVE-IL-1ra, pPAL7 KVVE-EL-1ra-KVVE, pPAL7 IL-1ra-KFFK, pPAL7 KFFK-IL-1ra, pPAL7 KFFK-IL-1ra-KFFK, pPAL7 IL-1ra-EFFE, pPAL7 EFFE-IL-1ra and pPAL7 EFFE-IL-1ra-EFFE were amplified in E. coli DH5α and purified using commercially available plasmid purification kit (Sigma). For expression, the above mentioned plasmids were cloned into E. coli BL21 (DE3) cells. Protein expression was induced using isopropyl-β-D-thiogalactopyranoside (IPTG) and the expressed protein was purified by affinity chromatography using Bio-scale mini Profinity eXact FPLC columns from Bio-Rad. Identity of the expressed protein was established by western blot using anti-IL-1ra antibody.

### Example 12

### Multimerisation of IL-1ra-KVVE, KVVE-IL-1ra and KVVE-IL-1ra-KVVE

Multimerisation of IL-1ra-KVVE, KVVE-IL-1ra and KVVE-IL-1ra-KVVE was performed under isothermal conditions. 1 ml of 20 mg/ml IL-1ra-KVVE, KVVE-IL-1ra and KVVE-IL-1ra-KVVE in 50 mM sodium phosphate buffer pH 6.0 was aliquoted into a 2 ml microcentrifuge tube and kept at 37° C. with shaking at 200 rpm. Kinetics of multimerisation was followed by measuring optical density (OD) at 405 nm at every 30 min interval caused by increase in turbidity.

### Example 13

### Multimerisation of KFFK-IL-1ra and EFFE-IL-1ra, IL-1ra-KFFK and IL-1ra-EFFE, KFFK-IL-1ra-KFFK and EFFE-IL-1ra-EFFE

Equimolar solutions of IL-1ra variants KFFK-IL-1ra and EFFE-IL-1ra, IL-1ra-KFFK and IL-1ra-EFFE, KFFK-IL-1ra-KFFK and EFFE-IL-1ra-EFFE in 50 mM sodium phosphate buffer pH 6.0 was aliquoted into a 2 ml microcentrifuge tube and kept at 37° C. with shaking at 200 rpm. Kinetics of multimerisation was followed by measuring optical density (OD) at 405 nm at every 30 min interval caused by increase in turbidity.

Though the multimerising motif KVVE did bring about multimerisation of IL-1ra to some extent but the process was slow and not as pronounced and efficient as in the case of KFFE. Since KFFK and EFFE alone are not able to multimerise therefore, equimolar solutions of fusion proteins bearing KFFK and EFFE at either or both ends were co-incubated. From the observed multimerisation profile, KFFK and EFFE (at single end) did not bring about a noteworthy change in the multimerisation profile of IL-1ra while their presence at both ends led to the formation of a few multimers.

SEQ ID NO: 8: Amino acid sequence of KVVE-IL-1ra (156 a.a.; 17.58 kDa)
KVVERPSGRKSSKMQAFRIWDVNQKTFYLRNNQLVAGYLQGPNVNLEEKIDVVPIEPHALFLGIHGGK
MCLSCVKSGDETRLQLEAVNITDLSENRKQDKRFAFIRSDSGPTTSFESAACPGWFICTAMEADQPVSL
TNMPDEGVMVTKFYFQEDE
SEQ ID NO: 9: Amino acid sequence of IL-1ra-KVVE (156 a.a.; 17.58 kDa)
RPSGRKSSKMQAFRIWDVNQKTFYLRNNQLVAGYLQGPNVNLEEKIDVVPIEPHALFLGIHGGKMCLS
CVKSGDETRLQLEAVNITDLSENRKQDKRFAFIRSDSGPTTSFESAACPGWFLCTAMEADQPVSLTNMP
DEGVMVTKFYFQEDEEVVK
SEQ ID NO: 10: Amino acid sequence of KVVE-IL-1ra-KVVE (160 a.a.; 18.03 kDa)
KVVERPSGRKSSKMQAFRIWDVNQKTFYLRNNQLVAGYLQGPNVNLEEKIDVVPIEPHALFLGIHGGK
MCLSCVKSGDETRLQLEAVNITDLSENRKQDKRFAFIRSDSGPTTSFESAACPGWELCTAMEADQPVSL
TNMPDEGVMVTKFYFQEDEEVVK
SEQ ID NO: 11: Amino acid sequence of KFFK-IL-1ra (156 a.a.; 17.68 kDa)
KFFKRPSGRKSSKMQAFRIWDVNQKTFYLRNNQLVAGYLQGPNVNLEEKIDVVPIEPHALFLGIHGGK
MCLSCVKSGDETRLQLEAVNITDLSENRKQDKRFAFIRSDSGPTTSFESAACPGWFLCTAMEADQPVSL
TNMPDEGVMVTKFYFQEDE
SEQ ID NO: 12: Amino acid.sequence of IL-1ra-KFFK (156 a.a.; 17.68 kDa)
RPSGRKSSKMQAFRIWDVNQKTFYLRNNQLVAGYLQGPNVNLEEKIDVVPIEPHALFLGIHGGKMCLS
CVKSGDETRLQLEAVNITDLSENRKQDKRFAFIRSDSGPTTSFESAACPGWFLCTAMEADQPVSLTNMP
DEGVMVTKFYFQEDEKFFK
SEQ ID NO: 13: Amino acid sequence of KFFK-IL-1ra-KFFK (160 a.a.; 18.23 kDa)
KFFKRPSGRKSSKMQAFRIWDVNQKTFYLRNNQLVAGYLQGPNVNLEEKIDVVPIEPHALFLGIHGGK
MCLSCVKSGDETRLQLEAVNITDLSENRKQDKRFAFIRSDSGPTTSFESAACPGWFLCTAMEADQPVSL
TNMPDEGVMVTKFYFQEDEKFFK
SEQ ID NO: 14: Amino acid sequence of EFFE-IL-1ra (156 a.a.; 17.68 kDa)
EFFERPSGRKSSKMQAFRIWDVNQKTFYLRNNQLVAGYLQGPNVNLEEKIDVVPIEPHALFLGIHGGK
MCLSCVKSGDETRLQLEAVNITDLSENRKQDKRFAFIRSDSGPTTSFESAACPGWFLCTAMEADQPVSL
TNMPDEGVMVTKFYFQEDE
SEQ ID NO: 15: Amino acid sequence of IL-1ra-EFFE (156 a.a.; 17.68 kDa)
RPSGRKSSKMQAFRIWDVNQKTFYLRNNQLVAGYLQGPNVNLEEKIDVVPIEPHALFLGIHGGICMCLS
CVKSGDETRLQLEAVNITDLSENRKQDKRFAFIRSDSGPTTSFESAACPGWFLCTAMEADQPVSLTNMP
DEGVMVTKFYFQEDEEFFE
SEQ ID NO: 16: Amino acid sequence of EFFE-IL-1ra-EFFE (160 a.a.; 18.23 kDa)
EFFERPSGRKSSKMQAFRIWDVNQKTFYLRNNQLVAGYLQGPNVNLEEKIDVVPIEPHALFLGIHGGK
MCLSCVKSGDETRLQLEAVNITDLSENRKQDKRFAFIRSDSGPTTSFESAACPGWELCTAMEADQPVSL
TNMPDEGVMVTKFYFQEDEEFFE

This invention may be embodied in other forms or carried out in other ways without departing from the spirit or essential characteristics thereof. The present disclosure is therefore to be considered as in all aspects illustrate and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

1-31. (canceled)

32. A multimeric form of interleukin-1 receptor antagonist (IL-1ra) wherein IL-1ra is covalently attached to or otherwise associated with a multimerising motif selected from KFFE, KVVE, KFFK and EFFE at the N-terminal end, C-terminal end, or both the N and C-terminal ends of IL-1ra and wherein the multimeric IL-1ra is capable of inhibiting IL-1 receptor and/or antagonizing IL-1, comprises IL-1ra multimers that weakly bind to Thioflavin T and Congo-red dye, and releases active IL-1ra monomers.

33. The multimeric IL-1ra of claim 32 which releases biologically active IL-1ra monomers for at least 1 day in vivo.

34. The multimeric IL-1ra of claim 32 which releases biologically active IL-1ra monomers in vitro or in vivo for 3-10 days.

35. The multimeric IL-1ra of claim 32 wherein the multimerisation motif is KFFE or KVVE.

36. The multimeric IL-1ra of claim 35 which is selected from IL-1raK, KIL-1ra and KIL-1raK as set forth in SEQ ID NO: 1, SEQ ID NO: 2 or SEQ ID NO:3.

37. The multimeric IL-1ra of claim 32 wherein the multimerisation motif is KFFK or EFFE.

38. The multimeric IL-1ra of claim 32 having an amino acid sequence as set forth in any of SEQ ID NOs: 1-3 or 8-16.

39. The multimeric IL-1ra of claim 32 which releases IL-1ra monomers at a rate ranging from 1.1 to 6 μg/ml for 3 to 10 days in vivo.

40. The multimeric Il-1ra of claim 32 wherein a single dose of said interleukin-1 receptor antagonist multimers ranging from 50 to 300 mg/kg body weight upon administration to a subject in need thereof reduces inflammation by at least 40%.

41. A composition for treating, inhibiting and/or ameliorating inflammatory diseases or disorders, rheumatoid disease, autoinflammatory disorders or conditions resulting from adverse effects of Interleukin-1, wherein the composition comprises the multimeric IL-1ra of claim 32 and a pharmaceutically acceptable carrier, additive or diluent.

42. A composition for treating, inhibiting and/or ameliorating inflammatory diseases or disorders, rheumatoid disease, autoinflammatory disorders or conditions resulting from adverse effects of Interleukin-1, wherein the composition comprises one or more multimeric IL-1ra of claim 38 and a pharmaceutically acceptable carrier, additive or diluent.

43. The composition of claim 42 comprising one or more variant of IL-1ra having an amino acid sequence as set forth in any of SEQ ID NOs: 1-3.

44. The composition of claim 41 further comprising one or more additional therapeutic agent capable of modulating an arthritic, inflammatory, or immune condition or disease.

45. The composition of claim 44, wherein the additional therapeutic agent is selected from a group consisting of an IL-1 specific fusion protein, anti-TNF biologicals, Etanercept, Infliximab, Humira, Adalimumab, thalidomide, a steroid, a DMARD, Colchicines, IL-18 BP or a derivative, an IL-18-specific fusion protein, anti-IL-18, anti-IL-18 RI, anti-IL-18 Rβ, anti-IL-1 RI, and anti IL-1 Ab.

46. The composition of claim 41 formulated for administration intramuscularly, intradermally, subcutaneously or intraperitoneally to a subject in need thereof.

47. The composition of claim 41, wherein said composition is formulated for administration through a device capable of releasing said composition, wherein said device is selected from the group consisting of pumps, catheters, patches and implants.

48. A process of preparation of the multimeric form of claim 32 comprising
a. dissolving a peptide therapeutic or variant IL-1ra attached to a multimerisation motif at a temperature of about 25-50° C. in a solution having pH range of about 4 to 8; and
b. incubating the above for a period of about 6 to 48 hours with constant shaking to obtain therapeutic insoluble and aggregated multimeric form of peptide therapeutic IL-1ra.

49. The process of claim 48 further comprising
c. washing the resulting multimers with PBS or another physiologically relevant or acceptable solvent or solution; and
d. resuspending said multimers in PBS or another physiologically relevant or acceptable solvent or solution.

50. The process of claim 48, wherein said solution is selected from a group consisting of sodium acetate buffer having pH in the range of about 3.5 to 5.5, sodium phosphate buffer, potassium phosphate buffer and phosphate buffer (PBS) having pH in the range of about 6-8 and citrate buffer in the range of about 4-6.

51. The process of claim 48, wherein the temperature ranges from 30-50° C., preferably about normal human body temperature or about 37° C.

52. A method of treating, inhibiting, and/or ameliorating inflammatory diseases or disorders, rheumatoid disease, autoinflammatory disorders or conditions resulting from adverse effects of Interleukin-1, wherein said method comprises administering a therapeutic amount of the multimeric form of IL-1ra of claim 32 to a subject in need.

53. The method of claim 52 further comprising administering one or more additional therapeutic agent capable of modulating an arthritic, inflammatory, or immune condition or disease.

54. The method of claim 53, wherein the additional therapeutic agent is selected from a group consisting of an IL-1 specific fusion protein, anti-TNF biologicals, Etanercept, Infliximab, Humira, Adalimumab, thalidomide, a steroid, a DMARD, Colchicines, IL-18 BP or a derivative, an IL-18-specific fusion protein, anti-IL-18, anti-IL-18 RI, anti-IL-18 Rβ, anti-IL-1 RI, and anti IL-1 Ab.

55. The method of claim 52 wherein the inflammatory disease or autoinflammatory disorder is arthritis, Inflammatory Bowel Disease, Ulcerative Colitis or acute hepatic injury.

56. The method of claim 55 wherein the arthritis is Rheumatoid Arthritis, Osteoarthritis, Psoriatic Arthritis, Ankylosing spondylitis or Juvenile Rheumatoid Arthritis.


SEQUENCE LISTING (43 sequences)

SEQ ID NO: 1 - protein, 156 aa, Artificial Sequence
  IL-1raK
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDEEFFK

SEQ ID NO: 2 - protein, 156 aa, Artificial Sequence
  KIL-1ra
KFFERPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 3 - protein, 160 aa, Artificial Sequence
  KIL-1raK
KFFERPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDEEFFK

SEQ ID NO: 4 - protein, 152 aa, Homo sapiens
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 5 - protein, 159 aa, Artificial Sequence
  IL-1raG
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDEGNNQQNY

SEQ ID NO: 6 - protein, 159 aa, Artificial Sequence
  GIL-1ra
GNNQQNYRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 7 - protein, 166 aa, Artificial Sequence
  GIL-1raG
GNNQQNYRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDEGNNQQNY

SEQ ID NO: 8 - protein, 156 aa, Artificial Sequence
  KVVE-IL-1ra
KVVERPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 9 - protein, 156 aa, Artificial Sequence
  IL-1ra-KVVE
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDEEVVK

SEQ ID NO: 10 - protein, 160 aa, Artificial Sequence
  KVVE-IL-1ra-KVVE
KVVERPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDEEVVK

SEQ ID NO: 11 - protein, 156 aa, Artificial Sequence
  KFFK-IL-1ra
KFFKRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 12 - protein, 156 aa, Artificial Sequence
  IL-1ra-KFFK
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDEKFFK

SEQ ID NO: 13 - protein, 160 aa, Artificial Sequence
  KFFK-IL-1ra-KFFK
KFFKRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDEKFFK

SEQ ID NO: 14 - protein, 156 aa, Artificial Sequence
  EFFE-IL-1ra
EFFERPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 15 - protein, 156 aa, Artificial Sequence
  IL-1ra-EFFE
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDEEFFE

SEQ ID NO: 16 - protein, 160 aa, Artificial Sequence
  EFFE-IL-1ra-EFFE
EFFERPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDEEFFE

SEQ ID NO: 17 - protein, 153 aa, Artificial Sequence
  Met-IL-1ra (anakinra)
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 18 - protein, 4 aa, Unknown
  multimerisation motif
KFFE

SEQ ID NO: 19 - protein, 4 aa, Unknown
  multimerisation motif
KVVE

SEQ ID NO: 20 - protein, 4 aa, Unknown
  multimerisation motif
KFFK

SEQ ID NO: 21 - protein, 4 aa, Unknown
  multimerisation motif
EFFE

SEQ ID NO: 22 - protein, 7 aa, Unknown
  multimerisation motif
GNNQQNY

SEQ ID NO: 23 - protein, 7 aa, Unknown
  multimerisation motif
KLVFFAE

SEQ ID NO: 24 - protein, 5 aa, Unknown
  multimerisation motif
NGAIL

SEQ ID NO: 25 - protein, 4 aa, Unknown
  multimerisation motif
NFLV

SEQ ID NO: 26 - protein, 5 aa, Unknown
  multimerisation motif
FLVHS

SEQ ID NO: 27 - protein, 8 aa, Unknown
  multimerisation motif
NFGSVQFV

SEQ ID NO: 28 - protein, 5 aa, Unknown
  multimerisation motif
DFNKF

SEQ ID NO: 29 - protein, 4 aa, Unknown
  multimerisation motif
DFNK

SEQ ID NO: 30 - protein, 7 aa, Unknown
  multimerisation motif
KLVFFAE

SEQ ID NO: 31 - protein, 33 aa, Unknown
  multimerisation motif
RMKQLEDKVEELLSKKYHLENEVARLKKLVGER

SEQ ID NO: 32 - DNA, 41 bp, Artificial Sequence
  KIL-1raK forward primer
aagctttgaaattttttgaacgaccctctgggagaaaatcc

SEQ ID NO: 33 - DNA, 46 bp, Artificial Sequence
  KIL-1raK reverse primer
aattcttatttaaaaaattcctcgtcctcctggaagtagaatttgg

SEQ ID NO: 34 - DNA, 458 bp, Homo sapiens
cgaccctctgggagaaaatccagcaagatgcaagccttcagaatctgggatgttaaccagaagaccttctatctgaggaacaaccaactagttgctggatattgcaaggaccaaatgtcaatttagaagaaaagatagatgtggtacccattggcctcatgctctgttcttgggaatccatggagggaagatgtgcctgtcctgttcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagta

SEQ ID NO: 35 - DNA, 12 bp, Unknown
  multimerising motif nucleic acid
aaattttttgaa

SEQ ID NO: 36 - DNA, 50 bp, Artificial Sequence
  GIL-1raG forward primer
aagctttgggcaacaaccaacaaaactatcgaccctctgggagaaaatcc

SEQ ID NO: 37 - DNA, 55 bp, Artificial Sequence
  GIL-1raG reverse primer
aattcttaatagttttgttggttgttgccctcgtcctcctggaagtagaatttgg

SEQ ID NO: 38 - DNA, 41 bp, Artificial Sequence
  KVVE-IL-1ra forward primer
aagctttgaaagtggtggaacgaccctctgggagaaaatcc

SEQ ID NO: 39 - DNA, 46 bp, Artificial Sequence
  KVVE-IL-1ra reverse primer
aattcttatttcaccacttcctcgtcctcctggaagtagaatttgg

SEQ ID NO: 40 - DNA, 41 bp, Artificial Sequence
  KFFK-IL-1ra forward primer
aagctttgaaattttttaaacgaccctctgggagaaaatcc

SEQ ID NO: 41 - DNA, 46 bp, Artificial Sequence
  KFFK-IL-1ra reverse primer
aattcttatttaaaaaatttctcgtcctcctggaagtagaatttgg

SEQ ID NO: 42 - DNA, 41 bp, Artificial Sequence
  EFFE-IL-1ra forward primer
aagctttggaattttttgaacgaccctctgggagaaaatcc

SEQ ID NO: 43 - DNA, 46 bp, Artificial Sequence
  EFFE-IL-1ra reverse primer
aattcttattcaaaaaattcctcgtcctcctggaagtagaatttgg